SEQUENCE LISTING (32 sequences)

SEQ ID NO: 1 - protein, 294 aa, Staphylococcus aureus
MADSDINIKTGTTDIGSNTTVKTGDLVTYDKENGMHKKVFYSFIDDKNHNKKLLVIRTKGTIAGQYRVYSEEGANKSGLAWPSAFKVQLQLPDNEVAQISDYYPRNSIDTKEYMSTLTYGFNGNVTGDDTGKIGGLIGANVSIGHTLKYVQPDFKTILESPTDKKVGWKVIFNNMVNQNWGPYDRDSWNPVYGNQLFMKTRNGSMKAADNFLDPNKASSLLSSGFSPDFATVITMDRKASKQQTNIDVIYERVRDDYQLHWTSTNWKGTNTKDKWTDRSSERYKIDWEKEEMTN

SEQ ID NO: 2 - DNA, 39 bp, Artificial Sequence
  Description of Artificial Sequence Synthetic oligonucleotide
mmmmmmmmmmmmmmmmmmmmmmmmmmmmmmmmmmmmmmm

SEQ ID NO: 3 - DNA, 40 bp, Artificial Sequence
  Description of Artificial Sequence Synthetic oligonucleotide
cccccccccccccccccccccccccccccccccccccccc

SEQ ID NO: 4 - DNA, 39 bp, Artificial Sequence
  Description of Artificial Sequence Synthetic oligonucleotide
ccccccccacccccccccccccccccccccccccccccc

SEQ ID NO: 5 - DNA, 39 bp, Artificial Sequence
  Description of Artificial Sequence Synthetic oligonucleotide
cccccccccccccccccaccccccccccccccccccccc

SEQ ID NO: 6 - DNA, 39 bp, Artificial Sequence
  Description of Artificial Sequence Synthetic oligonucleotide
cccccccccccccccccccaccccccccccccccccccc

SEQ ID NO: 7 - DNA, 40 bp, Artificial Sequence
  Description of Artificial Sequence Synthetic oligonucleotide
aaaaaaaaaaaaaaaaaaaaaaaaaaaaaaaaaaaaaaaa

SEQ ID NO: 8 - DNA, 40 bp, Artificial Sequence
  Description of Artificial Sequence Synthetic oligonucleotide
aaaaaaaaaaggggaaaaaaaaaaaaaaaaaaaaaaaaaa

SEQ ID NO: 9 - DNA, 100 bp, Artificial Sequence
  Description of Artificial Sequence Synthetic polynucleotide
cccccccccccccccccccccccccccccccccccccccccccccccccccccccccccccccccccccccccccccccccccccccccccccccccccc

SEQ ID NO: 10 - DNA, 36 bp, Artificial Sequence
  Description of Artificial Sequence Synthetic oligonucleotide
aaaaaaaaaaaaaaaaaaaaaaaaaaaaaaaaaaaa

SEQ ID NO: 11 - DNA, 40 bp, Artificial Sequence
  Description of Artificial Sequence Synthetic oligonucleotide
aaaaaaaaaagaaaaaaaaaaaaaaaaaaaaaaaaaaaaa

SEQ ID NO: 12 - DNA, 120 bp, Artificial Sequence
  Description of Artificial Sequence Synthetic polynucleotide
aaaaaaaaaaaaaaaaaaaaaaaaaaaaaaaaaaaaaaaacccccccccccccccccccccccccccccccccccccccccccccccccccccccccccccccccccccccccccccccc

SEQ ID NO: 13 - DNA, 80 bp, Artificial Sequence
  Description of Artificial Sequence Synthetic oligonucleotide
cccccccccccccccccccccccccccccccccccccccccccccccccccccccccccccccccccccccccccccccc

SEQ ID NO: 14 - DNA, 40 bp, Artificial Sequence
  Description of Artificial Sequence Synthetic oligonucleotide
aaaaaaaaaagggaaaaaaaaaaaaaaaaaaaaaaaaaaa

SEQ ID NO: 15 - DNA, 81 bp, Artificial Sequence
  Description of Artificial Sequence Synthetic oligonucleotide
aaaaaaaaaaaaaaaaaaaaaaaaaaaaaaaaaaaaaaaagaaaaaaaaaaaaaaaaaaaaaaaaaaaaaaaaaaaaaaaa

SEQ ID NO: 16 - DNA, 40 bp, Artificial Sequence
  Description of Artificial Sequence Synthetic oligonucleotide
aagaaaaaaaaaaaaaaaaaaaaaaaaaaaaaaaaaaaaa

SEQ ID NO: 17 - DNA, 40 bp, Artificial Sequence
  Description of Artificial Sequence Synthetic oligonucleotide
aaagaaaaaaaaaaaaaaaaaaaaaaaaaaaaaaaaaaaa

SEQ ID NO: 18 - DNA, 40 bp, Artificial Sequence
  Description of Artificial Sequence Synthetic oligonucleotide
aaaagaaaaaaaaaaaaaaaaaaaaaaaaaaaaaaaaaaa

SEQ ID NO: 19 - DNA, 40 bp, Artificial Sequence
  Description of Artificial Sequence Synthetic oligonucleotide
aaaaagaaaaaaaaaaaaaaaaaaaaaaaaaaaaaaaaaa

SEQ ID NO: 20 - DNA, 40 bp, Artificial Sequence
  Description of Artificial Sequence Synthetic oligonucleotide
aaaaaaagaaaaaaaaaaaaaaaaaaaaaaaaaaaaaaaa

SEQ ID NO: 21 - DNA, 40 bp, Artificial Sequence
  Description of Artificial Sequence Synthetic oligonucleotide
aaaaaaagaaaagaaaaaaaaaaaaaaaaaaaaaaaaaaa

SEQ ID NO: 22 - DNA, 40 bp, Artificial Sequence
  Description of Artificial Sequence Synthetic oligonucleotide
aaaaaaaaagggaaaaaaaaaaaaaaaaaaaaaaaaaaaa

SEQ ID NO: 23 - DNA, 39 bp, Artificial Sequence
  Description of Artificial Sequence Synthetic oligonucleotide
ccccccccccccccccccccccccccccccccccccccc

SEQ ID NO: 24 - DNA, 44 bp, Artificial Sequence
  Description of Artificial Sequence Synthetic oligonucleotide
aaaaaaaaaaaaaaaaaaaaaaaaaaaaaaaaaaaaaaaagggg

SEQ ID NO: 25 - DNA, 40 bp, Artificial Sequence
  Description of Artificial Sequence Synthetic oligonucleotide
rrrrrrrrrrrrrrrrrrrrrrrrrrrrrrrrrrrrrrrr

SEQ ID NO: 26 - DNA, 40 bp, Artificial Sequence
  Description of Artificial Sequence Synthetic oligonucleotide
aaggggaaaaaaaaaaaaaaaaaaaaaaaaaaaaaaaaaa

SEQ ID NO: 27 - DNA, 40 bp, Artificial Sequence
  Description of Artificial Sequence Synthetic oligonucleotide
aaaggggaaaaaaaaaaaaaaaaaaaaaaaaaaaaaaaaa

SEQ ID NO: 28 - DNA, 40 bp, Artificial Sequence
  Description of Artificial Sequence Synthetic oligonucleotide
aaaaggggaaaaaaaaaaaaaaaaaaaaaaaaaaaaaaaa

SEQ ID NO: 29 - DNA, 40 bp, Artificial Sequence
  Description of Artificial Sequence Synthetic oligonucleotide
aaaaaggggaaaaaaaaaaaaaaaaaaaaaaaaaaaaaaa

SEQ ID NO: 30 - DNA, 40 bp, Artificial Sequence
  Description of Artificial Sequence Synthetic oligonucleotide
aaaaaaaaaaaaaaaaaaaaggggaaaaaaaaaaaaaaaa

SEQ ID NO: 31 - DNA, 100 bp, Artificial Sequence
  Description of Artificial Sequence Synthetic polynucleotide
aaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 32 - DNA, 40 bp, Artificial Sequence
  Description of Artificial Sequence Synthetic oligonucleotide
tttttttttttttttttttttttttttttttttttttttt

(19) United States
(12) Patent Application Publication
(10) Pub. No.: US 2014/0248608 A1
(43) Pub. Date: Sep. 4, 2014

(54) METHODS FOR CHARACTERIZING A DEVICE COMPONENT BASED ON A CONTRAST SIGNAL TO NOISE RATIO

(71) Applicants: Geoffrey A. Barrall, San Diego, CA (US); Eric N. Ervin, Salt Lake City, UT (US); Prithwish Pal, San Diego, CA (US)

(72) Inventors: Geoffrey A. Barrall, San Diego, CA (US); Eric N. Ervin, Salt Lake City, UT (US); Prithwish Pal, San Diego, CA (US)

(73) Assignee: ELECTRONIC BIOSCIENCES, INC., San Diego, CA (US)

(21) Appl. No.: 14/128,588

(22) PCT Filed: Jun. 22, 2012
(86) PCT No.: PCT/US12/43864

§ 371 Date: May 9, 2014

Related U.S. Application Data
(60) Provisional application No. 61/500,971, filed on Jun. 24, 2011.
(60) Provisional application No. 61/513,439, filed on Jul. 29, 2011.
(60) Provisional application No. 61/513,458, filed on Jul. 29, 2011.

(51) Int. Cl. C12Q 1/68

(52) U.S. Cl. CPC C12Q 1/6869
USPC 435/6.1

(57) ABSTRACT

The general concept of using a nanopore for DNA sequencing is to electrophoretically drive a polymer (e.g. single stranded DNA) through a nanopore under aqueous conditions, and identify each individual monomer (e.g. nucleotide) of the strand as it passes through the sensitive region of the nanopore based on its characteristic current modulation.

### RELATED PATENT APPLICATION

This patent application is a national stage application of international patent application no. PCT/US2012/043864, filed on Jun. 22, 2012, entitled METHODS FOR CHARACTERIZING A DEVICE COMPONENT BASED ON A CONTRAST SIGNAL TO NOISE RATIO, naming Geoffrey A. Barrall, Eric N. Ervin, and Prithwish Pal as inventors, and having attorney docket no. EBS-1005-PC, which claims the benefit of U.S. Provisional Patent Application No. 61/513,458 filed on Jul. 29, 2011, entitled METHODS FOR CHARACTERIZING A NANOPORE BASED ON A CONTRAST SIGNAL TO NOISE RATIO naming Geoffrey Barrall, Eric N. Ervin, and Prithwish Pal as inventors, and designated by attorney docket no. EBS-1005-PV. This patent application also claims the benefit of U.S. provisional patent application No. 61/621,378 filed Apr. 6, 2012, entitled HIGH CONTRAST SIGNAL TO NOISE RATIO NANOPORES naming Geoffrey Barrall, Eric N. Ervin, and Prithwish Pal as inventors, and designated by attorney docket no. EBS-1003-PV3, U.S. provisional patent application No. 61/513,439 filed Jul. 29, 2011, entitled HIGH CONTRAST SIGNAL TO NOISE RATIO NANOPORES naming Geoffrey Barrall, Eric N. Ervin, and Prithwish Pal as inventors, and designated by attorney docket no. EBS-1003-PV2 and U.S. provisional patent application No. 61/500,971 filed Jun. 24, 2011, entitled HIGH CONTRAST SIGNAL TO NOISE RATIO NANOPORES naming Geoffrey Barrall, Eric N. Ervin, Prithwish Pal, as inventors, and designated by attorney docket no. EBS-1003-PV. The entire content of each of the foregoing provisional patent applications is incorporated herein by reference, including all text, tables and drawings.

### STATEMENT OF GOVERNMENT SUPPORT

This invention was made with government support under Contract No. 1R01HG005095 awarded by the National Institutes of Health, specifically the National Human Genome Research institute, and Contract No. HSHQDC-09-C-00091 awarded by the Department of Homeland Security. The government has certain rights in the invention.

### SEQUENCE LISTING

The instant application contains a Sequence Listing which has been submitted in ASCII format via EFS-Web and is hereby incorporated by reference in its entirety. Said ASCII copy, created on Aug. 21, 2012, is named EBS1005PC.txt and is 11,107 bytes in size.

### FIELD

The technology relates in part to methods of characterizing a device component based on a contrast signal to noise ratio.

### SUMMARY

The general concept of using a nanopore for DNA sequencing is to electrophoretically drive a polymer (e.g. single stranded DNA) through a nanopore under aqueous conditions, and identify each individual monomer (e.g. nucleotide) of the strand as it passes through the sensitive region of the nanopore based on its characteristic current modulation.

Using a low noise measurement apparatus (G. A. Barrall, R. C. Dunnam, M. A. Krupka et al., “Quartz Nanopore Membranes for Low Noise Measurements of Ion Channel Conductance,” Biophysical Journal, vol. 98, no. 3, Supplement 1, pp. 598a-598a, 2010.), it was determined that as single stranded DNA is translocating a nanopore, excess noise is produced that is sufficient to mask the transition between nucleotides. This excess noise appears as a variation in the pore current and scales with solution conductivity and the applied bias. The discovery of the excess noise produced as a polymer translocates through a nanopore suggests that a high contrast signal to noise ratio (CNR) nanopore is required to sequence a polymer.

It has been discovered that modifications to the protein pore itself can significantly reduce the excess noise produced by the DNA while maintaining or even increasing the contrast between nucleotides. It has been shown that the CNR can be affected with mutagenesis modifications. While high CNR is necessary to sequence, in some embodiments, it is not necessarily sufficient. However, methods to fully characterize a nanopore can be time consuming (i.e. mapping). The majority of nanopores modified typically do not have a high enough CNR for sequencing, thus further and more time consuming characterization of these nanopores becomes labor intensive. Provided herein are methods that can be used to rapidly characterize mutated nanopores based on their measured CNR.

Methods described herein determine a characterization contrast signal to noise ratio (CCNR) to characterize a nanopore, where the contrast signal is computed between a first level within a residual current measurement as a first composition section of a first polymer is located within a nanopore and a second level within a residual current measurement as a second composition section of a second polymer is located within the nanopore, and wherein the noise is a noise value associated with at least one of the levels used to compute the contrast signal used to compute the CCNR of the nanopore. In certain embodiments, the first composition section and the second composition section each are in different polymers, and sometimes the first composition section and the second composition section are within the same polymer.

In some embodiments a method for characterizing a nanopore is based on a characterization contrast signal to noise ratio (CCNR) comprising: measuring a first level within a residual current for a first composition section of a polymer; measuring a second level within a residual current for a second composition section of a polymer; calculating a contrast signal as a function of the first level and the second level; computing a noise value; calculating a characterization contrast signal to noise ratio (CCNR) as a function of the contrast signal and the noise value; and characterizing the nanopore based on the CCNR. In some embodiments the method comprises a first polymer comprising the first composition section and the second composition section. In some embodiments the method comprises a first polymer comprising the first composition section and a second polymer comprising the second composition section.

In some embodiments the method comprises a nanopore comprising a pore. In some embodiments the nanopore comprises a beta barrel. In some embodiments the nanopore is chosen from alpha hemolysin, MspA, OmpF, PA63 and gramicidin A. In some embodiments the nanopore is an alpha hemolysin.

In some embodiments a polymer is a protein or peptide. In some embodiments each polymer is a protein or peptide. In some embodiments a polymer is a nucleic acid. In some embodiments each polymer is a protein or peptide. In some embodiments the nucleic acid is chosen from DNA and RNA. In some embodiments the nucleic acid is single stranded or double stranded. In some embodiments the polymer is single stranded DNA. In some embodiments the composition section comprises at least a part of a monomer. In some embodiments the monomer independently is chosen from a nucleotide, monophosphate nucleotide, oxidized nucleotide, methylated nucleotide and modified nucleotide. In some embodiments the nucleotide comprises a base chosen from adenine, cytosine, thymine, guanine and uracil. In some embodiments each polymer is immobilized within the nanopore while the residual current is measured. In some embodiments each polymer is immobilized within the nanopore by attaching at least one molecule to the polymer. In some embodiments the at least one molecule attached to each polymer is chosen from biotin, tetraethylene glycol, avidin, streptavidin, Neutravidin and combinations thereof. In certain embodiments, the polymer is immobilized within the nanopore by having a portion of the polymer that is double stranded DNA to prevent translocation. In certain embodiments, the double stranded DNA can occur as a result of a hairpin, where the DNA self hybridizes. In certain embodiments, a complementary strand can be bound to a portion of the polymer to create a double stranded DNA portion. In some embodiments each polymer is within the nanopore and translocates the nanopore while the residual current is measured.

In some embodiments the first level is correlated to the composition of the first composition section of the polymer. In some embodiments the second level is correlated to the composition of the second composition section of the polymer. In some embodiments the composition section is a homopolymer. In some embodiments the composition section is a heteropolymer. In some embodiments calculating the contrast signal comprises determining the difference between a single measurement of the first level and a single measurement of the second level.

In some embodiments calculating the contrast signal comprises determining the difference between an average of the measurements of the first level and an average of the measurements of the second level. In some embodiments the average of the measurements of the first level is the average of at least 20 measurements of the first level. In some embodiments the average of the measurements of the second level is the average of at least 20 measurements of the second level. In some embodiments the noise value is the larger value of the noise computed for the first level and the noise computed for the second level. In some embodiments the noise value is the smaller value of the noise computed for the first level and the noise computed for the second level.

In some embodiments the noise level is computed for the first level and the second level. In some embodiments computing the noise value comprises using the noise amplitude at a given frequency. In some embodiments computing the noise value comprises using the root mean square noise value of the first level and the second level at a set filter frequency. In some embodiments the noise value is the square root of the sum of the squares of the standard deviation value for the first level and the standard deviation value for the second level at a set filter frequency. In some embodiments the noise value is extracted from the noise power spectral density.

In some embodiments the CCNR is calculated by dividing the contrast signal by the noise value. In some embodiments the residual current measurements are acquired using a Direct Current (DC) measurement system. In some embodiments the residual current measurements are acquired using an Alternating Current (AC) measurement system. In some embodiments data is filtered to a set filter frequency using a low pass filter. In some embodiments the data is filtered to a set filter frequency using a 3-pole Bessel low pass filter. In some embodiments the set filter frequency is chosen from about 1 Hz to about 500 Hz. In some embodiments the set filter frequency is about 300 Hz. In some embodiments the set filter frequency is about 10 kHz. In some embodiments characterizing the nanopore comprises comparing the CCNR calculated to a threshold CCNR. In some embodiments the set threshold CCNR is 2. In some embodiments the threshold CCNR is 5. In some embodiments a nanopore for which a calculated CCNR is equal to or greater than the threshold CCNR is subjected to further testing. In some embodiments the further testing comprises mapping the nanopore. In some embodiments the mapping comprises contacting the nanopore with heteropolymers. In some embodiments the heteropolymers comprise a particular nucleotide or nucleotide sequence at a different position in each of the heteropolymers. In some embodiments the mapping comprises computing the CCNR for the heteropolymers.

In some embodiments characterizing the nanopore comprises determining the effect of a mutagenesis modification on the CCNR computed for the nanopore. In some embodiments characterizing the nanopore comprises identifying one or more further mutagenesis modifications that can be made to the nanopore. In some embodiments characterizing the nanopore comprises selecting a nanopore that can determine the sequence of a polymer. In some embodiments the polymer is a nucleic acid. In some embodiments a device comprises a nanopore characterized by any one of the methods disclosed herein.

Certain embodiments are described further in the following description, examples, claims and drawings.

### BRIEF DESCRIPTION OF THE DRAWINGS

The drawings illustrate embodiments of the technology and are not limiting. For clarity and ease of illustration, the drawings are not made to scale and, in some instances, various aspects may be shown exaggerated or enlarged to facilitate an understanding of particular embodiments.

FIG. 1 shows a plot or map of the percent difference of the residual current of poly(C39)(A)X (SEQ ID NO: 2) compared to polyC40 (SEQ ID NO: 3) for wild type alpha hemolysin and the mutated alpha hemolysin termed 4SL135I.

FIG. 2 shows a plot of histogram values for wild type alpha hemolysin for the polymers polyC39AX (where X is position 9 (SEQ ID NO: 4), 18 (SEQ ID NO: 5) and 20 (SEQ ID NO: 6) in this plot) and polyC40 (SEQ ID NO: 3).

FIG. 3 shows data from capture and release experiments of biotinylated polyA40 (SEQ ID NO: 7) attached to streptavidin.

FIG. 4 shows a plot of the absolute contrast between A and C versus the RMS noise level for numerous alpha hemolysin pores characterized.

### DETAILED DESCRIPTION

### Polymers and Monomers

In one embodiment, the nanopores, modified nanopores, devices and methods described herein detect polymers or portions thereof. In some embodiments the nanopores, devices and methods described herein detect the monomeric units (e.g. monomers) that make up a polymer. In some embodiments the nanopores, devices and methods described herein detect the sequence of monomeric units (e.g. nucleotides) that make up a polymer (e.g. a strand of DNA or RNA).

A polymer, as referred to herein, can be any molecular polymer. Some common molecular polymers are polynucleotides and polypeptides. A polymer can be a nucleic acid polymer, a protein polymer or a peptide polymer. A polymer can be a single stranded or double stranded nucleic acid. A polymer can be a single stranded or double stranded DNA or RNA. A polymer can be a protein or peptide. Non-limiting examples of a polymer include a single stranded DNA, a double stranded DNA, a single stranded RNA, a double stranded RNA, a protein and a peptide. In certain embodiments, the polymer is single stranded DNA. In certain embodiments, the polymer is double stranded DNA. A polymer can include one or more sections and a polymer section can include at least a portion of a monomer.

A monomer, as referred to herein, can be any molecule that may bind chemically to another molecule to form a polymer. A monomer can be naturally occurring, modified or synthetic. A monomer can be a nucleic acid or amino acid, for example. A nucleic acid monomer can be phosphorylated, oxidized, acetylated, methylated or sulfonated. A nucleic acid monomer can be a monophosphate nucleotide, modified nucleotide, methylated nucleotide, acetylated nucleotide or oxidized nucleotide. Non-limiting examples of a monomer include nucleotides, monophosphate nucleotides, modified nucleotides, methylated nucleotides and oxidized nucleotides. Non-limiting examples of a nucleic acid monomer include adenine (A), cytosine (C), thymine (T), guanine (G), uracil (U), modified cytosine, 7-methylguanine, xanthine, hypoxanthine, 5,6-dihydrouracil, 5-methylcytosine, N4-methylcytosine, hydroxymethylcytosine, or N6-methyladenine.

As an example, sequences of single stranded DNA are noted according to the following format. For a polymer that is single stranded DNA and is comprised of 10 adenine (A) bases followed by 4 guanine (G) bases and then 26 adenine (A) bases, it will be written as polyA10G4A26 (SEQ ID NO: 8) for example. As another example, 100 cytosine (C) bases in single stranded DNA will be written as polyC100 (SEQ ID NO: 9).

A polymer can include one or more polymer sections and sometimes a first polymer includes a first polymer section and a second polymer includes a second polymer section. In some embodiments, a first polymer comprises at least one composition section and a second polymer comprises at least one composition section. Each composition section comprises at least a part of one monomer. The determination of a composition section is based upon it being comprised of at least a different type of at least a part of a monomer than another composition section. For example, in certain embodiments, the first polymer is polyA40 (SEQ ID NO: 7) and the second polymer is polyC40 (SEQ ID NO: 3). In this example, the first composition section is A40 (SEQ ID NO: 7), which is associated with the first level I_A40 (“A40” disclosed as SEQ ID NO: 7), and the second composition section is C40 (SEQ ID NO: 3), which is associated with the second level I_C40 (“C40” disclosed as SEQ ID NO: 3). A “level” as used herein can refer to a measurement of electrical current, a change in current, any current value or any value derived mathematically from a current value. A current level (e.g., in picoamps) for a section can be denoted as I_A36 (“A36” disclosed as SEQ ID NO: 10) (for A36 (SEQ ID NO: 10)), or I_G4 (for G4), for example. In another example, the first polymer is comprised of polyA40 (SEQ ID NO: 7), and the second polymer is comprised of polyA10GA29 (SEQ ID NO: 11), where the composition section is G in the latter polymer. In certain embodiments, when the first polymer and the second polymer are the same polymer, the polymer is comprised of at least two composition sections. For example, in certain embodiments, the polymer is polyA40C80 (SEQ ID NO: 12), where the composition sections of the single stranded DNA are A40 (SEQ ID NO: 7) and C80 (SEQ ID NO: 13).

In certain embodiments, it is not mandatory that a composition section is comprised of only one type of monomer. In the example of polyACAA, the composition sections could be AC and AA, since the two composition sections have at least one monomer type that is different. In another example, it is possible that the monomers comprising the composition section are not contiguous. For example, with the single stranded DNA polyACTG, it is possible that the composition sections could include the combination of A and T as well as another composition section being comprised of C and G. Such polymers can be used when a nanopore has two or more sensitive regions that are separated within the nanopore and that when combined produce the measured residual current.

### Homopolymers

In certain embodiments, the sequences of the polymers are known. In certain embodiments, the sequence of the polymer is known and the polymers are homopolymers (e.g. polyA40 (SEQ ID NO: 7) or polyC40 (SEQ ID NO: 3)) and thus the level of the residual current is known to be correlated to the composition of the polymer. For example, when measuring the residual current of a nanopore as a polymer is located within it, different levels can occur that are due to effects such as artifacts and protein gating. These levels, if used as one of the discrete levels to calculate the CCNR, would produce a CCNR that is not relevant to a nanopore's ability to be used in a polymer sequencing system. However, when using a homopolymer, it is straightforward to determine what level is associated with the actual composition of the polymer rather than a potential artifact because only a single monomer type is present.

### Heteropolymers

In certain embodiments, homopolymers are not used to compute the CCNR of the nanopore. In certain embodiments, a polymer is immobilized within a nanopore. In certain embodiments, a polymer is immobilized within a nanopore and at least one of the polymers is a heteropolymer. For example, the CCNR can be computed for the polymers polyA40 (SEQ ID NO: 7) and polyA10GA29 (SEQ ID NO: 11), where both polymers are measured when immobilized within the nanopore. In this example, the CCNR is computed between composition sections A40 (SEQ ID NO: 7) and G.

In certain embodiments, the nanopore has a sensitive region that is defined as the region or regions of the nanopore that is responsible for producing the majority of the residual current measurement. In certain embodiments, the sensitive region of the nanopore is known and the position of the target monomer within a heteropolymer can be chosen based on this sensitive region without additional testing. For example, if a nanopore had a sensitive region at position 11, then the polymers polyA10GA29 (SEQ ID NO: 11) and polyA40 (SEQ ID NO: 7) could be used to compute the CCNR between composition sections A40 (SEQ ID NO: 7) and G. In another example, if the nanopore has a sensitive region at positions 11-13, the polymers polyA10G3A27 (SEQ ID NO: 14) and polyA40 (SEQ ID NO: 7) could be used to compute the CCNR between composition sections A40 (SEQ ID NO: 7) and G3.

In certain embodiments, the sensitive region of the nanopore is unknown. In certain embodiments, in order to identify the location(s) of the sensitive region(s), the nanopore can be mapped with a single monomer within a homopolymer comprised of a different monomer, for example with the polymer polyA40GA40 (SEQ ID NO: 15). In certain embodiments, in order to map the nanopore, a single monomer, in this example the nucleotide containing the G base, is moved down a strand of DNA one position at a time such that the G base occupies each position that is within the nanopore during the residual current measurement.

For example, with the nanopore example of the protein pore wild type alpha hemolysin, the following sequences (TABLE 1) could be used to map the nanopore.

TABLE 1

| NAME | SEQUENCE |
|---|---|
| BtnTg-5′- | BiotinTEG-5′-AAGAAAAAAAAAAAAAAAAA-A20- |
| (A39)(G)3 | 3′ (SEQ ID NO: 16) |
| BtnTg-5′- | BiotinTEG-5′-AAAGAAAAAAAAAAAAAAAA-A20- |
| (A39)(G)4 | 3′ (SEQ ID NO: 17) |
| BtnTg-5′- | BiotinTEG-5′-AAAAGAAAAAAAAAAAAAAA-A20- |
| (A39)(G)5 | 3′ (SEQ ID NO: 18) |
| BtnTg-5′- | BiotinTEG-5′-AAAAAGAAAAAAAAAAAAAA-A20- |
| (A39)(G)6 | 3′ (SEQ ID NO: 19) |

In TABLE 1, BtnTg-5′-(A39)(G)3 (SEQ ID NO: 16) indicates that there is a Biotin TEG molecule on the 5′ end of the single stranded DNA. There is one G base that is moved down the strand of DNA and the 3 indicates that the G base is located 3 bases from the TEG molecule on the 5′ end. For the example of wild type alpha hemolysin, this could continue through the G base being placed in the 21^{st} position. In certain embodiments, the positions that show a difference from the background (e.g. BtnTg-5′-(A40 (SEQ ID NO: 7))) are indicated as sensitive regions within the nanopore. In certain embodiments, this position(s) can be chosen to place the target monomer(s) that are to be used to compute the CCNR. For example, a nanopore is mapped and found to have a sensitive region at position 8. The polymers polyA7GA32 (SEQ ID NO: 20) and polyA40 (SEQ ID NO: 7) can then be used to compute the CCNR between A and G. In another example, a nanopore is found to have sensitive regions at positions 8 and 13. In this case, the polymers polyA7GA4GA27 (SEQ ID NO: 21) and polyA40 (SEQ ID NO: 7) can be used to compute the CCNR between A and G. In certain embodiments, a nanopore has a sensitive region(s) that is defined by two or more positions. For example, a nanopore is found to have a sensitive region at positions 10-12. In this case, the polymers polyA9G3A28 (SEQ ID NO: 22) and polyA40 (SEQ ID NO: 7) can be used to compute the CCNR between A and G.

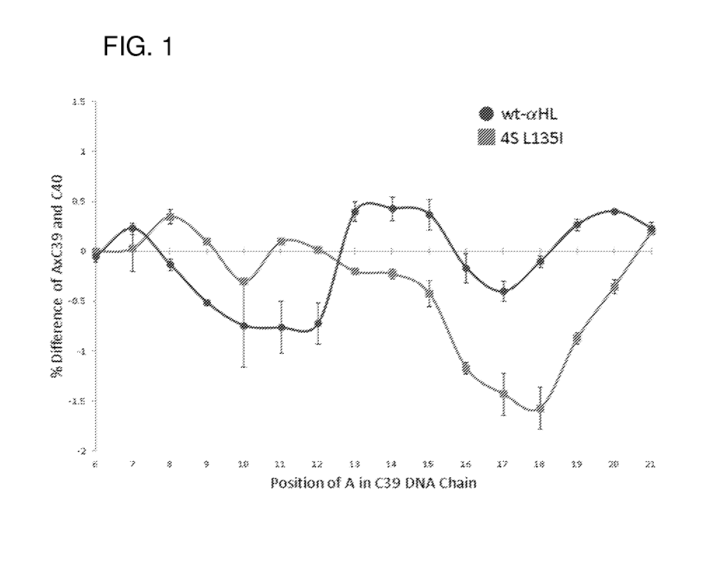

An example of the mapping of a nanopore is shown in FIG. 1. A single adenine base in a polyC39 (SEQ ID NO: 23) background was moved down the strand of DNA from position 6 to position 21 relative to the Biotin TEG molecule on the 5′ end. The data was taken in 3 M NaCl with a 120 mV applied DC bias and the DNA was immobilized in the pore using biotin TEG. The polymer was BtnTg-5′-poly(C39)(A)X (SEQ ID NO: 2), where X is the position of the A base relative to the Biotin TEG. FIG. 1 shows the maps for two nanopores, wild type alpha hemolysin (wt-αHL) and the alpha hemolysin mutant where the native amino acids E111, M113, K147 and T145 were all changed to serine and the L135 amino acid was changed to isoleucine (45L135I). FIG. 1 shows the percent difference of the residual current of poly(C39)(A)X (SEQ ID NO: 2) (where X notes the position number) compared to a polyC40 (SEQ ID NO: 3) polymer. For wild type alpha hemolysin, it can be seen that there are sensitive regions at positions 10-12, 13-15, and 17. For the 4SL135I mutant, the sensitive regions are at positions 8, 10, and 16-19.

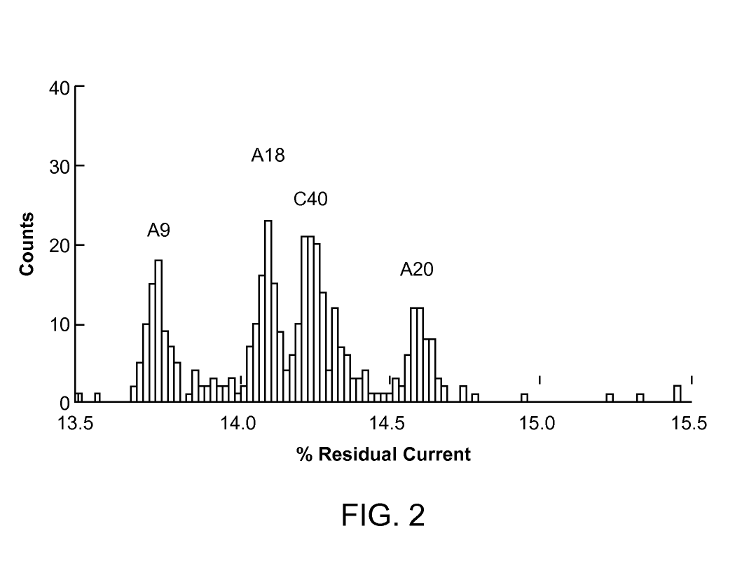

The values shown in FIG. 1 for the % differences of polyC39AX (SEQ ID NO: 2) compared to polyC40 (SEQ ID NO: 3) are obtained by plotting histograms of the residual current values for individual measurements. Examples of these plots can be seen in FIG. 2 for wild type alpha hemolysin. The mean value computed from the histogram is the data point plotted in FIG. 1 for each position and the error bars denote the spread in the histogram plots.

### Correlation Using Polymers to Map the Nanopore

In certain embodiments, if the CCNR is computed using immobilized polymers (e.g., heteropolymers or homopolymers), the levels are directly correlated to the composition of the composition section of the polymer.

In certain embodiments, if translocating heteropolymers are used to compute the CCNR, the levels can be correlated to the composition sections of the polymer by using a heteropolymer to map the nanopore in a similar manner as that used to compute the CCNR for immobilized heteropolymers. For example, the polymer polyA40G4 (SEQ ID NO: 24) is allowed to translocate the nanopore and the CCNR is computed between the level for the A40 (SEQ ID NO: 7) composition section and the G4 composition section. In certain embodiments, the nanopore can be mapped using the polymer BtnTg-5′-(A36)(G4)X (SEQ ID NO: 25), where X is the position of the first G base in the polymer and where the G4 composition section is moved down the polymer to each position that resides within the nanopore. In this example, X can equal any whole number from 1 to 37 (e.g., denoting the position of G4 at any of the positions from position 1 to position 37). For the example polymer BtnTg-5′-(A36)(G4)X (SEQ ID NO: 25), A36 (SEQ ID NO: 10) indicates the polymer comprises 36 A residues (SEQ ID NO: 10) and G4 indicates the polymer comprises 4 G residues. Examples of some polymer sequences that can be used when the nanopore is wild type alpha hemolysin are shown in TABLE 2.

TABLE 2

| NAME | SEQUENCE |
|---|---|
| BtnTg-5′- | BiotinTEG-5′-AAGGGGAAAAAAAAAAAAAA-A20- |
| (A36)(G4)3 | 3′ (SEQ ID NO: 26) |
| BtnTg-5′- | BiotinTEG-5′-AAAGGGGAAAAAAAAAAAAA-A20- |
| (A36)(G4)4 | 3′ (SEQ ID NO: 27) |
| BtnTg-5′- | BiotinTEG-5′-AAAAGGGGAAAAAAAAAAAA-A20- |
| (A36)(G4)5 | 3′ (SEQ ID NO: 28) |
| BtnTg-5′- | BiotinTEG-5′-AAAAAGGGGAAAAAAAAAAA-A20- |
| (A36)(G4)6 | 3′ (SEQ ID NO: 29) |
| BtnTg-5′- | BiotinTEG-5′-A20-GGGGAAAAAAAAAAAAAAAA- |
| (A36)(G4)21 | 3′ (SEQ ID NO: 30) |

In certain embodiments, the residual current value with the largest difference from the background composition section is compared to the measured level within the translocating measurements in order to determine whether the measured level correlates to the composition of the composition section of the polymer rather than a measurement artifact.

### Correlation Using Translocating Homopolymers

In certain embodiments, the CCNR is measured using a heteropolymer that translocates the nanopore, for example polyA40C80 (SEQ ID NO: 12). In this example, the first composition section and second composition section are within the same polymer, wherein the first composition section would be associated with a level for A40 (SEQ ID NO: 7), I_A40 (“A40” disclosed as SEQ ID NO: 7), and the second composition section would be associated with a level for C80 (SEQ ID NO: 13), I_C80 (“C80” disclosed as SEQ ID NO: 13). In certain embodiments, the correlation of the discrete levels to the composition of the composition sections of the polymer can be done by allowing homopolymers (e.g. polyA100 (SEQ ID NO: 31) or polyC100 (SEQ ID NO: 9)), to translocate through the nanopore and measure the residual current. The homopolymers would include the monomers that are expected or known to produce the levels within the heteropolymer (A40 (SEQ ID NO: 7) and C80 (SEQ ID NO: 13) in this example) being measured to compute the CCNR.

The homopolymers to be measured to correlate the levels to the composition of the composition sections of the heteropolymer could be polyA100 (SEQ ID NO: 31) and polyC100 (SEQ ID NO: 9) for example. In certain embodiments, both the homopolymers and heteropolymer can be present in the same electrolyte solution that is being measured to compute the CCNR. This minimizes the variation that is known to occur between nanopores of the same composition and experimental setups. The residual current values measured for polyA100 (SEQ ID NO: 31) and polyC100 (SEQ ID NO: 9) do not have to exactly equal the respective levels seen within the heteropolymer polyA40C80 (SEQ ID NO: 12) for example. However, in certain embodiments, it is expected that the absolute difference between levels within the heteropolymer and the absolute difference between the values for the two homopolymers will be approximately equal or substantially similar (within about 30%) for the same measurement conditions. For example, the I_A40 (“A40” disclosed as SEQ ID NO: 7), within polyA40C80 (SEQ ID NO: 12) might be 49 pA and the I_C80 (“C80” disclosed as SEQ ID NO: 13) is 61 pA, giving an absolute difference of 12 pA. The residual current for polyA100 (SEQ ID NO: 31) might be 47 pA current and the residual current for polyC100 (SEQ ID NO: 9) might be 59 pA, again providing an absolute difference of about 12 pA and thus approximately the same contrast signal. In another example, the residual current level for polyA100 (SEQ ID NO: 31) might be 49.4 pA and the residual current for polyC100 (SEQ ID NO: 9) might be 58.5 pA, producing a contrast signal of 9.1 pA. While this is a lower contrast signal than seen between I_A40 (“A40” disclosed as SEQ ID NO: 7) and I_C80 (“C80” disclosed as SEQ ID NO: 13), the value is sufficiently close and the relative blocking levels have the same relative magnitude (e.g. polyA100 (SEQ ID NO: 31) blocks more than polyC100 (SEQ ID NO: 9)), that the levels can be correlated to the composition of the polymer.

### Normalized Current

It is known that the current of a nanopore can vary per experiment based on the exact experimental conditions. As a result, it may be necessary to normalize the residual current values compared to the open channel current value of the nanopore. The normalized current value is the quotient of the residual current divided by the open channel current. Thus, the levels within the residual current measured as the polymer translocates the nanopore may be correlated to the values measured for the homopolymers based on the normalized values. In certain embodiments, these normalized values are used for the correlation of the levels to the actual composition of the composition sections of the polymer rather than being used in the calculation of the CCNR.

### Contrast Signal

In certain embodiments, a residual current measurement is made as a first composition section of a first polymer is located within the nanopore. In certain embodiments, a residual current measurement is made as a second composition section of a second polymer is located within the nanopore. A residual current measurement is a measurement of the reduced current that occurs as a polymer is blocking a nanopore. In certain embodiments, the first polymer and the second polymer are the same polymer.

In certain embodiments, the first composition section of the first polymer within the nanopore produces a first level within the residual current measurement and the second composition section of the second polymer within the nanopore produces a second level within the residual current measurement. In certain embodiments, at least one measurement of each polymer is taken. In certain embodiments, one to 10,000 measurements or greater of each polymer is taken (e.g. 1 or greater, 10 or greater, 20 or greater, 30 or greater, 40 or greater, 50 or greater, 60 or greater, 70 or greater, 80 or greater, 90 or greater, 100 or greater, 200 or greater, 300 or greater, 400 or greater, 500 or greater, 600 or greater, 700 or greater, 800 or greater, 900 or greater, 1,000 or greater, 5,000 or greater, or 10,000 or greater).

In certain embodiments, the polymer is translocating the nanopore when measuring the residual current through the nanopore. In some embodiments, the polymer is immobilized within the nanopore while measuring the residual current through the nanopore. In certain embodiments, the polymer is immobilized within the nanopore by attaching at least one molecule to the polymer that is unable to translocate through the nanopore. Non-limiting examples of a molecule for this use include biotin, tetraethylene glycol, avidin, streptavidin, Neutravidin and combinations thereof. In certain embodiments, the polymer is immobilized within the nanopore by having a portion of the polymer that is double stranded DNA to prevent translocation. In certain embodiments, the double stranded DNA can occur as a result of a hairpin, where the DNA self hybridizes. In certain embodiments, a complementary strand can be bound to a portion of the polymer to create a double stranded DNA portion. In some embodiments a complementary strand of single stranded nucleic acid hybridized to a portion of each polymer.

The contrast signal often is computed as a function of the first level and the second level. A contrast signal can be computed by taking the difference between the first level and the second level (e.g., subtracting the first level from the second level, subtracting the second level from the first level, dividing the first level by the second level, or dividing the second level by the first level), or any other suitable arithmetic function based on the first level and the second level that is informative of the contrast signal. In certain embodiments, the contrast signal is computed between an individual measurement of the first level and an individual measurement of the second level. In certain embodiments, the contrast signal is computed between an average, mean or median of the measurements for the first level and an average, mean or median of the measurements for the second level. In certain embodiments, the contrast signal is the average, mean or median of the differences between the first level and the second level for two or more measurements.

In certain embodiments, the first level and the second level have approximately the same value and thus the contrast signal is zero. For example, if immobilized polyA40 (SEQ ID NO: 7) and immobilized polyC40 (SEQ ID NO: 3) both produce a residual current measurement of 20 pA in a nanopore, then the contrast signal is zero and there is no contrast between polyA40 (SEQ ID NO: 7) and polyC40 (SEQ ID NO: 3). In another example, if the single stranded DNA polymer polyA40C80 (SEQ ID NO: 12) is allowed to translocate a nanopore, and only one discrete level is seen, the contrast signal is again zero as the value for the first level for composition section A40 (SEQ ID NO: 7) cannot be distinguished from the value for the second level for composition section C80 (SEQ ID NO: 13).

### Noise Value

In certain embodiments, the noise value is related to a computation of the noise for the first level. In certain embodiments, the noise value is related to a computation of the noise for the second level. In certain embodiments, the noise value is related to a computation of the noise for the first level and a computation of the noise for the second level.

The noise associated with either of the levels can be computed in numerous ways. Any known method of computing the noise of a measurement or a quantity correlated to the noise of a measurement can be used in this application.

In certain embodiments, the noise value is the variance of the residual current at a set filter frequency associated with the first level, the second level or both. The noise value can be any variation or computation of the noise associated with the variance of the residual current such as the standard deviation or the square root of the sum of the squares. In certain embodiments, the noise value is the amplitude of the noise power spectral density at a given frequency of the residual current associated with the first level, the second level or both. In certain embodiments the noise value is the integral of the power spectral density up to a given frequency of the residual current associated with the first level, the second level or both. The noise power spectral density may be computed by any known method, including but not limited to the use of Fourier transforms, Welch's method, maximum entropy, autoregression, and band pass filtering. Variations on the noise power spectral density such as the square root of the noise power spectral density may be used in place of the noise power spectral density. In certain embodiments, the noise value is the larger noise value between the noise for the first level and the noise for the second level. In certain embodiments, the noise value is the smaller noise value between the noise for the first level and the noise for the second level. In certain embodiments, the noise value is a combination of noise values associated with both the first and second levels. In certain embodiments the noise value is the square root of the sum of the squares of the noise values associated with the first and second levels.

In certain embodiments, the noise value is computed by taking the standard deviation of the residual current associated with the first level at a set filter frequency. In certain embodiments, the noise is computed by taking the standard deviation of the residual current associated with the second level at a set filter frequency. In certain embodiments, the larger noise value of the standard deviation value for the first level and the standard deviation value for the second level is used to compute the CCNR. In certain embodiments, the smaller noise value of the standard deviation value for the first level and the standard deviation value for the second level is used to compute the CCNR. In certain embodiments, the square root of the sum of the squares for the standard deviation value for the first level and the standard deviation value for the second level is used to compute the CCNR at a set filter frequency.

### Nanopores

In some embodiments, a nanopore is a protein pore. In some embodiments, a nanopore comprises one or more proteins or polypeptide subunits. In certain embodiments, a protein pore includes a pore shaped, or shaped in part, by one or more beta barrels. Non-limiting examples of a protein pore include the beta barrel containing transmembrane proteins including the bacterial porins alpha hemolysin (e.g., from Staphylococcus aureus), MspA (e.g., from Mycobacterium smegmatis), OmpF (E. coli), PA63 (B. anthracis), and gramicidin A (B. brevis). In some embodiments a nanopore comprises a porin protein. In some embodiments a nanopore comprises a hemolysin protein. In some embodiments, a protein pore can be embedded in a lipid bilayer by known methods.

### Porin Proteins

Porin proteins are proteins that fall into the beta-barrel class of transmembrane proteins. Porins act as a pore or channel through which molecules can diffuse. Unlike other membrane transport proteins, porins are large enough to allow passive diffusion. Porins typically control the diffusion of small metabolites, like sugars, ions, amino acids and the like. Porins sometimes are chemically selective. A particular porin sometimes transports only one group of molecules, and sometimes a porin is specific for only one molecule.

Porins typically are composed of beta sheets, which in turn, generally are linked together by beta turns on the cytoplasmic side and long loops of amino acids on the other side. The beta sheets lie in an anti-parallel fashion and form a cylindrical tube, called a beta barrel. The amino acid composition of the porin beta sheets is unique in that polar and nonpolar residues alternate along the beta sheets. This arrangement results in a conformation in which most or all of the nonpolar residues typically face outward so as to interact with the nonpolar lipid membrane, and most or all of the polar residues typically face inwards into the center of the beta barrel to interact with the aqueous channel.

Non-limiting examples of porin proteins include aquaporins (e.g., from mammals and plants), maltoporins and other sugar specific porins (e.g., from gram negative bacteria (e.g., E. coli, S. typhimurium, and other bacteria), OmpG porin (e.g., gram negative bacteria), opacity porins (e.g., from Neisseria bacteria), nucleoside specific porin (e.g., from E. coli and other bacteria), MspA porin (e.g., from Mycobacterium smegmatis), gramicidin (e.g., Bacillis brevis), and alpha hemolysin (e.g., from Staphylococcus Aureus).

### Hemolysins and Alpha-Hemolysin

Hemolysins are exotoxins produced by bacteria that cause lysis of red blood cells in vitro or in vivo. Visualization of hemolysis of red blood cells in agar plates facilitates the categorization of some pathogenic bacteria such as Streptococcus and Staphylococcus. Although the lytic activity of some hemolysins on red blood cells may be important for nutrient acquisition or for causing certain conditions such as anemia, many hemolysin-producing pathogens do not cause significant lysis of red blood cells during infection. Although hemolysins are able to lyse red blood cells in vivo, the ability of hemolysins to target other cells, including white blood cells, often accounts for the effects of hemolysins in the host. Many hemolysins are pore forming proteins.

A non-limiting example of a porin protein useful for insertion into lipid bilayers is alpha-HL, sometimes also referred to as alpha toxin. Alpha-hemolysin (e.g., alpha-HL) forms a heptameric beta-barrel in biological membranes. Alpha-HL is secreted as a monomer that binds to the outer membrane of susceptible cells. Upon binding, the monomers oligomerize to form a water-filled transmembrane channel that facilitates uncontrolled permeation of water, ions, and small organic molecules. Rapid discharge of vital molecules, such as ATP, dissipation of the membrane potential and ionic gradients, and irreversible osmotic swelling leading to rupture or lysis of the cell wall, frequently causing death of the host cell. This pore-forming property has been identified as a major mechanism by which protein toxins cause damage to cells.

Several properties of alpha-HL make this membrane channel suitable for various biotechnological applications: assembled alpha-HL is stable over a wide range of pH and temperature, its transmembrane pore stays open at normal conditions, alpha-HL can insert into to various biological or synthetic lipid bilayers, the insertion proceeds spontaneously and does not require specific ionic conditions. Alpha-HL inserted into a lipid bilayer may prove useful as delivery systems, as a component of a stochastic sensor, and transporters or translocators. Alpha-HL has been shown to have the ability to translocate nucleic acids through the pore formed in a lipid bilayer.

Non-limiting examples of pore forming hemolysins include listeriolysin O (e.g., from Listeria monocytogenes), alpha toxin or alpha hemolysin (e.g., from Staphylococcus aureus), PVL cytotoxin (e.g., from Staphylococcus aureus), and cytolysin A (e.g., from E. coli).

### Modifications of a Nanopore

In certain embodiments, the protein pore contains at least one mutagenesis modification. In certain embodiments, the at least one mutagenesis modification includes substituting at least one native amino acid within the protein pore. In certain embodiments, the at least one mutagenesis modification to the protein pore can include substituting at least one native amino acid to a non-native amino acid that modifies the contrast signal. In certain embodiments, the at least one mutagenesis modification to the protein pore can include substituting at least one native amino acid with a non-native amino acid that modifies the total noise value. In certain embodiments, the at least one mutagenesis modification to the protein pore can include substituting at least one native amino acid with a non-native amino acid that modifies the CCNR. In certain embodiments, the mutagenesis modification can include substituting a native amino acid with a naturally occurring amino acid or a synthetic amino acid. In certain embodiments, the synthetic amino acid can include chemically synthesized amino acids with non-natural side-chains.

While one or more mutagenesis modifications may modify or remove a pore structure of a protein pore, the mutated protein still is referred to as a “nanopore” or “protein pore” when the protein to which the mutations are introduced includes a pore.

In certain embodiments, the protein pore is alpha hemolysin. Alpha hemolysin can include a primary constriction. The primary constriction can include at least the native amino acids E111, M113, K147 and T145. Alpha hemolysin can include a secondary constriction. The secondary constriction can include at least the native amino acids N121, G137, N139, L135, N123, and T125. Alpha hemolysin can include an exit region. The exit region can include at least the native amino acids D127, T129 and K131. In some embodiments, alpha hemolysin can include one or more salt bridges. In some embodiments, a salt bridge includes a pair of native amino acids E111 and K147 and/or the pair of amino acids D127 and K131.

The at least one modification to an alpha hemolysin pore can include substituting at least one of the native amino acids in the primary constriction in the alpha hemolysin pore to simplify the primary constriction. Non-limiting examples of a substitution to simplify the primary constriction include an amino acid that reduces the charge compared to the native amino acid, an amino acid that eliminates the charge of the side chain of the native amino acid, an amino acid that reduces the hydrogen bonding between the amino acid and the polymer compared to the native amino acid and the polymer, an amino acid that is smaller in size than the native amino acid, and an amino acid that changes the hydrophobic interactions between the amino acid and the DNA. The substitution to simplify the primary constriction can increase the contrast signal, reduce the total noise value or both. In certain embodiments, the substitution to simplify the primary constriction can increase the CCNR.

The at least one modification to the alpha hemolysin pore can include substituting at least one of the native amino acids in the secondary constriction to enhance the secondary constriction. Non-limiting examples of a substitution to enhance the secondary constriction include an amino acid that changes the charge compared to the native amino acid, an amino acid that increases hydrogen bonding between the amino acid and the polymer compared to the native amino acid and the polymer, an amino acid that changes the hydrophobic interactions between the amino acid and the DNA, and an amino acid that is larger in size than the native amino acid. The substitution to enhance the secondary constriction can increase the contrast signal, reduce the total noise value or both. In certain embodiments, the substitution to enhance the secondary constriction can increase the CCNR.

In some embodiments, an at least one modification to an alpha hemolysin pore includes substituting at least one of the native amino acids in a salt bridge to a different amino acid to change, disrupt, add or move the salt bridge. In certain embodiments, a substitution to a salt bridge is changing one of the native amino acids in a salt bridge to the same charge as the other amino acid in the salt bridge (e.g. changing D127 (negative) to lysine (K, positive), which is the same charge as K131). In certain embodiments, a substitution to a salt bridge is changing one of the native amino acids in a salt bridge to a different amino acid with the same charge (e.g. changing E111 (negative) to aspartic acid (D), which is also negatively charged). In certain embodiments, at least one of the native amino acids is changed to introduce a charge and salt bridge. The substitution to change or disrupt a salt bridge can increase the contrast signal, reduce the total noise value or both. In certain embodiments, the substitution to change or disrupt a salt bridge can increase the CNR.

The at least one modification to the alpha hemolysin pore can include a combination of substitutions to any of the native amino acids within the alpha hemolysin pore.

### Characterization Contrast Signal to Noise Ratio

The Characterization Contrast Signal to Noise Ratio (CCNR) often is computed as a function of the contrast signal and the noise value. A CCNR can be calculated as the difference between the contrast signal and the noise value (e.g., contrast signal subtracted from the noise value, noise value subtracted from the contrast signal, contrast signal divided by the noise value, and noise value divided by the contrast signal), or any other suitable arithmetic function based on the contrast signal and noise value that is informative of CCNR. In some embodiments, a method for characterizing a nanopore is based on a characterization contrast signal to noise ratio (CCNR). The term “based on” or “based from” as used herein can mean based in or derived from a mathematical or scientific premise. The term “based on” or “based from” as used herein can mean derived from a mathematical or scientific argument from which a conclusion is drawn. The term “based on” or “based from” as used herein can mean derived from mathematical or scientific data from which a conclusion is drawn. For example, a method for characterizing a nanopore can be derived from a CCNR. In some embodiments, a method for characterizing a nanopore is based on a method of obtaining a CCNR measurement that best characterizes a nanopore. In some embodiments, a method for characterizing a nanopore is based on a method of obtaining a CCNR measurement that determines a composition section of a polymer. In some embodiments, a method for characterizing a nanopore is based on a method of obtaining a CCNR measurement that most accurately predicts a composition section of a polymer. In some embodiments, a method for characterizing a nanopore is based on a CCNR measurement that most accurately predicts a composition section of a polymer.

### Characterization

The CCNR is used to characterize the nanopores being investigated. The CCNR is a critical parameter to being able to utilize a nanopore for polymer sequencing; however, it is not necessarily the only parameter that needs to be considered when identifying a nanopore useful for polymer sequencing. Methods described herein can be used to rapidly characterize nanopores useful for polymer sequencing applications based on a CCNR.

### Screening

In certain embodiments, the CCNR is used to characterize a nanopore wherein the characterization involves screening the nanopores for candidates for polymer sequencing or additional nanopore tests. In some embodiments characterizing a nanopore comprises selecting a nanopore that can determine the sequence of a polymer.

In some embodiments a polymer is used to characterize a nanopore. In some embodiments a method for characterizing nanopores based on a CCNR comprises the use of one or more polymers wherein each polymer is a protein or peptide. In some embodiments a method for characterizing nanopores based on a CCNR comprises the use of one or more polymers wherein each polymer is a nucleic acid polymer. The term “each polymer” as used herein can mean only one polymer if only one is used to characterize a nanopore. The term “each polymer” can mean one or more, or all polymers if more than one polymer is used to characterize a nanopore.

In certain embodiments, a nanopore is screened by setting a minimum threshold for the CCNR at a set filter frequency.

In some embodiments, the CCNR at the set filter frequency meets at least a set threshold value. In certain embodiments, the set threshold value is at least 2, at least 2.5, at least 3, at least 3.5, at least 4, at least 4.5, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 40, at least 50, at least 60, at least 70, at least 80, at least 90, or at least 100.

In some embodiments, the CCNR is computed and a nanopore is selected based on its CCNR compared to another nanopore's CCNR. For example, a native protein pore such as wild type alpha hemolysin might have a CCNR of 5 at a set filter frequency of 300 Hz. A mutated alpha hemolysin nanopore might be selected for additional testing based on the fact that it has a CCNR of 7 at a set filter frequency, for example.

In certain embodiments, the CCNR is given at the set filter frequency (df0). In some embodiments, the CCNR (CCNR0) can be scaled by frequency to obtain the approximately equivalent (within 30%) CCNR (CCNR1) at a different frequency (df1) according to the equation CCNR1=CCNR0*sqrt(df0/df1). For example, the CCNR can be measured at 300 Hz for ease of data processing and then be scaled to a frequency of 10 KHz to determine what the CCNR would be at a bandwidth that might be used in a polymer sequencing system.

In certain embodiments, the additional nanopore tests include mapping the nanopore according to methods as described in Purnell et al. and in this application, or computing the CCNR as polymers translocate through the nanopore (R. F. Purnell, K. K. Mehta, and J. J. Schmidt, “Nucleotide Identification and Orientation Discrimination of DNA Homopolymers Immobilized in a Protein Nanopore” Nano letters, Aug. 13, 2008). In some embodiments mapping a nanopore comprises computing the CCNR for heteropolymers.

In certain embodiments, additional nanopore tests include additional CCNR measurements with different polymers. For example, a nanopore could have its CCNR measured with polyA40 (SEQ ID NO: 7) and polyC40 (SEQ ID NO: 3) immobilized strands and if it meets a set threshold, the CCNR may then be tested with the immobilized polymers polyC40 (SEQ ID NO: 3) and polyT40 (SEQ ID NO: 32) as an additional test.

### Guiding Modifications

In certain embodiments, the CCNR is used to characterize the nanopore, wherein the characterization involves interpreting the effects of a modification made to the nanopore. In certain embodiments, the characterization involves guiding future nanopore modifications based on the CCNR. For example, a modified alpha hemolysin pore might be tested where a modification has been made to its primary constriction, such as substituting the methionine at the M113 position to serine to create the mutant termed M113S in this application. Based on the computed CCNR, additional modifications might be made to the nanopore that include the M113S substitution in addition to other substitutions or it might be determined that the M113S modification does not help the CCNR and thus is not tested in future modifications.

This method allows a relatively easy and quick test to be performed to determine a critical parameter, the CCNR, for a large number of nanopores to be able to assess if additional investigation is necessary for this particular mutation for polymer sequencing applications. In some embodiments, the CCNR is quantified under a range of measurement conditions (e.g. solution, temperature) in order to determine whether a modification to the measurement system is beneficial.

### Data Acquisition, Sampling Frequency, Effective Bandwidth and Set Filter Frequency

The following is provided as examples of measurement systems and acquisition parameters that can be used to obtain and filter the data in order to compute the CCNR of a nanopore. This information should in no way be considered limiting and any measurement system or data acquisition parameters can be used to compute the CCNR.

In certain embodiments, the residual current measurements can be acquired using a Direct Current (DC) measurement system. In certain embodiments, a DC bias, which is a substantially constant applied voltage, is applied across the nanopore. In certain embodiments, the residual current signal is low pass filtered with an analog filter prior to digitally acquiring the residual current data at an acquisition frequency. The resulting data is then digitally low pass filtered to an effective bandwidth and then resampled at a sampling frequency. In certain embodiments, the CCNR is computed at a set filter frequency. In certain embodiments, the set filter frequency is equal to the effective bandwidth. In certain embodiments, the CCNR can be computed at a set filter frequency that is lower than the effective bandwidth.

In certain embodiments, the residual current measurements can be acquired using an Alternating Current (AC) measurement system. In certain embodiments, the AC measurement system is the measurement system as described in U.S. Pat. No. 7,731,826, herein incorporated by reference. In certain embodiments, the AC system utilizes a source signal that is periodic (e.g. a sine wave or square wave) that is defined by an applied bias and frequency. In certain embodiments, the frequency of the source signal is equal to a center frequency. In certain embodiments, the source signal is applied and the residual current signal is low pass filtered with an analog filter prior to digitally acquiring the residual current data at an acquisition frequency. The resulting data is then demodulated as described in U.S. Pat. No. 7,731,826 to produce the effective DC measurement of the residual current. In certain embodiments, the data can be further low pass filtered to an effective bandwidth and resembled at a sampling frequency. In certain embodiments, the CCNR is computed at a set filter frequency. In certain embodiments, the set filter frequency is equal to the effective bandwidth. In certain embodiments, the CCNR can be computed at a set filter frequency that is lower than the effective bandwidth.

In certain embodiments, a DC bias is applied while acquiring the residual current measurements using the AC measurement system. In certain embodiments, the DC bias is in the range of about 1 mV to about 300 mV or greater (e.g. 1 mV, 2 mV, 3, mV, 4 mV, 5 mV, 6 mV, 7 mV, 8 mV, 9 mV, 10 mV, 15, mV, 20 mV, 25 mV, 30 mV, 35 mV, 40 mV, 45 mV, 50 mV, 60 mV, 70 mV, 80 mV, 90 mV, 100 mV, 110 mV, 120 mV, 130 mV, 140 mV, 150 mV, 160 mV, 170 mV, 180 mV, 190 mV, 200 mV, 210 mV, 220 mV, 230 mV, 240 mV, 250 mV, 260 mV, 270 mV, 280 mV, 290 mV or 300 mV). In certain embodiments, the DC bias is in the range of about 5 mV to about 300 mV (e.g. at least 5 mV, 6 mV, 7 mV, 8 mV, 9 mV, 10 mV, 15, mV, 20 mV, 25 mV, 30 mV, 35 mV, 40 mV, 45 mV, 50 mV, 60 mV, 70 mV, 80 mV, 90 mV, 100 mV, 110 mV, 120 mV, 130 mV, 140 mV, 150 mV, 160 mV, 170 mV, 180 mV, 190 mV, 200 mV, 210 mV, 220 mV, 230 mV, 240 mV, 250 mV, 260 mV, 270 mV, 280 mV, 290 mV or 300 mV or greater).

### Acquisition Frequency

In certain embodiments, the acquisition frequency is the rate at which the data is digitally acquired.

### Sampling Frequency

In certain embodiments, the sampling frequency is the final number of data points per second.

### Low Pass Filter

In certain embodiments, a low pass filter is a filter that passes low frequency signals, but attenuates signals with frequencies approaching and higher than the cutoff frequency. In certain embodiments, the low pass filter is defined by the cutoff frequency, the frequency at which the filter attenuates the input power by about 3 dB. For example, for a 10 kHz low pass filter, the cutoff frequency is 10 kHz and the filter attenuates the input power by about 3 dB at 10 kHz.

### Effective Bandwidth

The largest signal bandwidth that can be realized is defined as the Nyquist frequency, which is the sampling frequency divided by 2. In certain embodiments, the residual current signal or data is to be filtered using a low pass or band-pass filter to an effective bandwidth that is at or below the Nyquist frequency. In certain embodiments, the sampling frequency is 5 times greater than the effective bandwidth.

In certain embodiments, the low pass filtering can be done prior to acquiring the data at the acquisition frequency using an analog filter for anti-aliasing or after acquiring the data at the acquisition frequency using a digital filter. In certain embodiments, the residual current signal can be low pass filtered using an analog low pass filter for anti-aliasing and then low pass filtered again after acquiring the data at the acquisition frequency using a digital low pass filter.

### Set Filter Frequency for CCNR Calculation

In certain embodiments, the CCNR is calculated at a specific frequency, termed the set filter frequency or predetermined filter frequency, in this application. In certain embodiments, the set filter frequency is equal to the effective bandwidth. In certain embodiments, the data is further filtered with a low pass filter to the set filter frequency.

In certain embodiments, non-limiting examples of the set filter frequency are frequencies between 1 Hz and 500 kHz (e.g. 1 Hz, 10 Hz, 100 Hz, 200 Hz, 300 Hz, 400 Hz, 500 Hz, 600 Hz, 700 Hz, 800 Hz, 900 Hz, 1 kHz, 2 kHz, 3 kHz, 4 kHz, 5 kHz, 6 kHz, 7 kHz, 8 kHz, 9 kHz, 10 kHz, 20 kHz, 30 kHz, 40 kHz, 50 kHz, 100 kHz, 200 kHz, 300 kHz, 400 kHz and 500 kHz). In certain embodiments, the set filter frequency is 300 Hz. In certain embodiments, the set filter frequency is 10 kHz.

In certain embodiments, the signal is low pass filtered using an analog filter for anti-aliasing. In certain embodiments, the signal is low pass filtered using an analog 1-pole Bessel filter. The data is then digitally acquired at the acquisition frequency and then low pass filtered to the effective bandwidth. In certain embodiments, the data is low pass filtered to the effective bandwidth using a digital 8-pole Bessel filter. The data is then resembled at the sampling frequency. In certain embodiments, the sampling frequency is equal to the acquisition frequency. In certain embodiments, the sampling frequency is lower than the acquisition frequency. In certain embodiments, the CCNR is computed at a set filter frequency that equals the effective bandwidth. In certain embodiments, the CCNR is computed at a set filter frequency that is lower than that of the effective bandwidth. In certain embodiments, the CCNR is computed at a set filter frequency that is lower than the effective bandwidth by low pass filtering the data to the set filter frequency. In certain embodiments, the data is low pass filtered to the set filter frequency using a 3-pole Bessel filter.

As an example, the signal is low pass filtered with a 1-pole Bessel analog filter to 170 kHz for anti-aliasing. The data is then digitally acquired at an acquisition frequency of 1.25 MHz. Then the data is digitally low pass filtered to 50 kHz, the effective bandwidth, using a digital 8-pole Bessel filter. The data is then resampled at a sampling frequency of 250 kHz, which provides a Nyquist frequency of 125 kHz. The CCNR is to be calculated at a set filter frequency (300 Hz), which is lower than the effective bandwidth (100 kHz). The data is low pass filtered to the set filter frequency of 300 Hz using a 3-pole Bessel filter.

In certain embodiments, an AC source signal is applied and the residual current signal is low pass filtered using an analog filter for anti-aliasing. In certain embodiments, the signal is low pass filtered using an analog 1-pole Bessel filter. The data is then digitally acquired at the acquisition frequency. The data is then demodulated as described in U.S. Pat. No. 7,731,826 to produce the effective DC measurements of the residual currents. In certain embodiments, the data is low pass filtered using a digital filter to an effective bandwidth. In certain embodiments, the data is low pass filtered to the effective bandwidth using a digital 8-pole Bessel filter and resampled at the sampling frequency. In certain embodiments, the sampling frequency is equal to the AC source frequency. In certain embodiments, the CCNR is computed at a set filter frequency that equals the effective bandwidth. In certain embodiments, the CCNR is computed at a set filter frequency that is lower than that of the effective bandwidth. In certain embodiments, the CCNR is computed at a set filter frequency that is lower than the effective bandwidth by low pass filtering the data to the set filter frequency. In certain embodiments, the data is low pass filtered to the set filter frequency using a 3-pole Bessel filter.

As an example, a sinusoidal source signal of 100 mV and a frequency of 50 kHz is applied. The AC signal is low pass filtered using a 1-pole analog filter for anti-aliasing with a cutoff frequency of 175 kHz. The data is then digitally acquired at an acquisition frequency of 800 kHz. The data is then demodulated using the source signal to produce the effective DC measurements of the residual currents at a sampling frequency of 50 kHz (equal to the source frequency). The data is then digitally low pass filtered using an 8-pole Bessel filter to 300 Hz. The CCNR is then computed at a set filter frequency of 300 Hz, which is lower than the effective bandwidth.

### Nanopore Devices

In some embodiments, provided is a device or apparatus that includes a nanopore selected by a characterization method described herein. Any suitable device capable of supporting a nanopore and allowing for sensing of an analyte can be utilized. Nanopore devices are often comprised of a substrate that includes an aperture and one or more proteins inserted in the aperture. In certain embodiments, the protein is inserted in a lipid monolayer and/or bilayer that traverses the aperture. In some embodiments, the nanopore protein is retained within the aperture without a lipid monolayer and/or bilayer. In some embodiments, a substrate includes a well and one or more proteins inserted in the well opening within a lipid monolayer and/or bilayer that traverses the well opening. In certain embodiments, a substrate includes a well and one or more proteins inserted in the well opening without a lipid monolayer and/or bilayer that traverses the well opening.

In certain embodiments, the apparatus further comprises a DC measurement system. In some embodiments, the apparatus further comprises an AC measurement system. In certain embodiments, the apparatus further comprises an AC/DC measurement system.

In some embodiments, the substrate comprises glass, Si, SiO₂, Si₃N₄, alumina, nitrides, diamond, quartz, sapphire metals, ceramics, alumino-silicate, polymers (e.g., Teflon, polycarbonate), the like or combinations thereof. Non-limiting examples of glass types suitable for a substrate include fused silica glass, ninety-six percent silica glass, soda-lime silica glass, borosilicate glass, aluminosilicate glass, lead glass, doped glass comprising desired additives, functionalized glass comprising desired reactive groups, the like and combinations thereof. Non-limiting examples of minerals (e.g., quartz) suitable for a substrate include quartz, tridymite, cristobalite, coesite, lechatelierite, stishovite, the like and combinations thereof. The substrate can be manufactured from a pure substance or can be manufactured from a composite material.

The thickness of a substrate typically ranges from about 100 nanometer (nm) to 5 millimeters (mm) in thickness (e.g., about 100 nm, about 150 nm, about 200 nm, about 250 nm, about 300 nm, about 350 nm, about 400 nm, about 500 nm, about 600 nm, about 700 nm, about 800 nm, about 900 nm, about 1000 nm (e.g., about 1 μm), about 2 μm, about 3 μm, about 4 μm, about 5 μm, about 6 μm, about 7 μm, about 8 μm, about 9 μm, about 10 μm, about 15 μm, about 20 μm, about 25 μm, about 30 μm, about 35 μm, about 40 μm, about 45 μm, about 50 μm, about 60 μm, about 70 μm, about 80 μm, about 90 μm, 100 μm, about 110 μm, about 120 μm, about 130 μm, about 140 μm, about 150 μm, about 175 μm, about 200 μm, about 225 μm, about 250 μm, about 300 μm, about 350 μm, about 400 μm, about 450 μm, about 500 μm, about 600 μm, about 700 μm, about 800 μm, about 900 μm, 1000 μm (e.g. 1 mm), about 2 mm, about 3 mm, about, about 4 mm, or about 5 mm).

In certain embodiments, a substrate contains an aperture that separates two fluid reservoirs. In some embodiments, the aperture is a micron scale aperture, and sometimes the aperture is a nanoscale aperture. In some embodiments, the aperture is in a glass or quartz substrate. In certain embodiments, the aperture has a diameter of about 0.25 nanometer to about 100 μm (e.g., about 0.25 nanometers, about 0.5 nanometers, about 1 nanometer, about 1.5 nanometers, about 2 nanometers, about 2.5 nanometers, about 3 nanometers, about 3.5 nanometers, about 4 nanometers, about 4.5 nanometers, about 5 nanometers, about 6 nanometers, about 7 nanometers, about 8 nanometers, about 9 nanometers, about 10 nanometers, about 15 nanometers, about 20 nanometers, about 25 nanometers, about 30 nanometers, about 35 nanometers, about 40 nanometers, about 45 nanometers, about 50 nanometers, about 60 nanometers, about 70 nanometers, about 80 nanometers, about 90 nanometers, about 100 nanometers, about 125 nanometers, about 150 nanometers, about 175 nanometers, about 200 nanometers, about 250 nanometers, about 300 nanometers, about 350 nanometers, about 350 nanometers, about 400 nanometers, about 500 nanometers, about 600 nanometers, about 700 nanometers, about 800 nanometers, about 900 nanometers, about 1000 nanometers (e.g., 1 μm), about 2 μm, about 3 μm, about 4 μm, about 5 μm, about 10 μm, about 15 μm, about 20 μm, about 25 μm, about 30 μm, about 35 μm, about 40 μm, about 45 μm, or about 50 μm).

In certain embodiments, a substrate comprises a well. In some embodiments, the well has an aperture formed by the well opening with a diameter of about 100 nanometers to about 100 μm (e.g., about 100 nanometers, about 125 nanometers, about 150 nanometers, about 175 nanometers, about 200 nanometers, about 250 nanometers, about 300 nanometers, about 350 nanometers, about 350 nanometers, about 400 nanometers, about 500 nanometers, about 600 nanometers, about 700 nanometers, about 800 nanometers, about 900 nanometers, about 1000 nanometers (e.g., 1 μm), about 2 μms, about 3 μm, about 4 μm, about 5 μm, about 10 μm, about 15 μm, about 20 μm, about 25 μm, about 30 μm, about 35 μm, about 40 μm, about 45 μm, about 50 μm, about 60 μm, about 70 μm, about 80 μm, about 90 μm or about 100 μm).

The channel formed by the aperture in a substrate is of any suitable geometry, and sometimes has a substantially circular, oval, square, rectangular, rhomboid, parallelogram, or other like cross-section. The channel in the substrate is of any suitable profile, and sometimes has a substantially cylindrical or conical (e.g., tapering or expanding conical) profile.

A substrate sometimes comprises a coating that modifies the surface of an aperture or well structure. In some embodiments, a substrate comprises a surface that includes a hydrophobic substance. In certain embodiments, a substrate comprises a surface that includes a hydrophilic substance. In some embodiments, a substrate comprises a surface that includes hydrophobic and hydrophilic substances.

Thus, one or more portions of, or the entire, substrate can be treated or coated to adopt certain desirable characteristics, in some embodiments. In certain embodiments, the treatment or coating enhances formation of lipid structures across the aperture of the substrate. Physical and/or chemical modification of the surface properties of a substrate include, but are not limited to, modification of the electrical charge density, changes to the hydrophobicity, changes to the hydrophilicity, the like and combinations thereof. Any suitable substance can be utilized to modify one or more interior and/or exterior surfaces of the substrate. Non-limiting examples of suitable materials for modification of one or more substrate surfaces include silanes, silanes terminating in a cyano group, silanes terminating in a methyl group, thiols, the like, or combinations thereof. In some embodiments, an exterior surface of a substrate may be modified by a first entity. In certain embodiments, an interior surface of a substrate may be modified by a second entity. In some embodiments, the first and the second entity may be the same entities, and in certain embodiments, the first and the second entity may be different entities. In some embodiments utilizing a glass substrate, the first or second entities that can be used to modify the interior or exterior surfaces of a substrate include a variety of glass-reactive species, e.g., 3-cyano-propyldimethylchlorosilane, that react with the silanol groups of the glass surface.

In some embodiments, a device comprises a lipid composition (e.g., monolayer, bilayer, combination thereof) over, across or spanning an aperture of a substrate. A lipid composition sometimes comprises a lipid monolayer, sometimes comprises a lipid bilayer, and in some embodiments comprises a lipid layer that partially is a monolayer and partially is a bilayer. In some devices comprising both monolayer and bilayer lipid structures, solvent may be trapped at a location (i.e., annulus) between the substrate and the lipid layer at or near the monolayer and bilayer interface, which is addressed in greater detail hereafter.

The lipid composition of a device often is relatively stable to mechanical disturbances, and can have a lifetime in excess of two weeks. Additionally, a device can be made with a lipid composition that is readily formed over or in an aperture and has a relatively small surface area, which can give rise to favorable electrical characteristics.

Nanopore membrane devices can comprise a channel or nanopore embedded in a suitable material. The diameter of an aperture of a channel in a membrane, across which an amphiphilic composition forms in a nanopore membrane system, often ranges in diameter from about 0.25 nanometers to about 50 μm (e.g., about 0.25 nanometers, about 0.5 nanometers, about 1 nanometer, about 1.5 nanometers, about 2 nanometers, about 2.5 nanometers, about 3 nanometers, about 3.5 nanometers, about 4 nanometers, about 4.5 nanometers, about 5 nanometers, about 6 nanometers, about 7 nanometers, about 8 nanometers, about 9 nanometers, about 10 nanometers, about 15 nanometers, about 20 nanometers, about 25 nanometers, about 30 nanometers, about 35 nanometers, about 40 nanometers, about 45 nanometers, about 50 nanometers, about 60 nanometers, about 70 nanometers, about 80 nanometers, about 90 nanometers, about 100 nanometers, about 125 nanometers, about 150 nanometers, about 175 nanometers, about 200 nanometers, about 250 nanometers, about 300 nanometers, about 350 nanometers, about 350 nanometers, about 400 nanometers, about 500 nanometers, about 600 nanometers, about 700 nanometers, about 800 nanometers, about 900 nanometers, about 1000 nanometers (e.g., 1 μm), about 1.5 μm, about 2 μm, about 2.5 μm, about 3 μm, about 3.5 μm, about 4 μm, about 5 μm, about 10 μm, about 15 μm, about 20 μm, about 25 μm, about 30 μm, about 35 μm, about 40 μm, about 45 μm, or about 50 μm). The channel formed in the membrane is of any suitable geometry, and sometimes has a substantially circular, oval, square, rectangular, rhomboid, parallelogram, or other like cross-section. The channel formed in the membrane is of any suitable profile, and sometimes has a substantially cylindrical or conical (e.g., tapering or expanding conical) profile. Nanopore membrane devices often are composed of a single conical-shaped channel or nanopore embedded in a suitable material. Membranes can be formed as known in the art and as described herein.

While a device often comprises a lipid composition traversing a substrate aperture, the composition traversing the substrate aperture may comprise any suitable amphiphilic molecule(s) or material(s) that can stably traverse an aperture and into which a protein can be incorporated. An amphiphilic molecule generally is composed of a hydrophobic portion and a polar portion. The terms “amphiphilic material” or “amphiphilic materials” refer to materials made of molecules having a polar, water-soluble group attached to a nonpolar, water-insoluble hydrocarbon chain. Amphiphilic materials sometimes can be polymers. Amphiphilic materials may be a pure substance or a mixture of different amphiphilic materials. The polymeric materials may be a polymer with a uniform molecular weight distribution, or a polymer with a non-uniform molecular weight distribution, or a mixture of polymers which comprise different monomers. Non-limiting examples of amphiphilic materials include lipids, detergents, surfactants, proteins, polysaccharides, and other chemical or biochemical materials that can be rendered amphiphilic.

The terms “detergent” or “detergents” as used herein refer to a surfactant or a mixture of surfactants. In some embodiments, “surfactant” or “surfactants” refer to any compound that (i) lowers the surface tension of a liquid, allowing easier spreading, and/or (ii) lowers the interfacial tension between two liquids, or between a liquid and a solid. Surfactants may act as: detergents, wetting agents, emulsifiers, foaming agents, and dispersants. Surfactants often are categorized as ionic (anionic or cationic), zwitterionic or amphoteric, or non-ionic. Non-limiting examples of surfactants include ammonium lauryl sulfate, sodium lauryl sulfate (SDS), sodium laureth sulfate (e.g., also known as sodium lauryl ether sulfate (SLES)), sodium myreth sulfate, dioctyl sodium sulfosuccinate, perfluorooctanesulfonate (PFOS), perfluorobutanesulfonate, alkyl benzene sulfonates, alkyl aryl ether phosphate, alkyl ether phosphate, fatty acid salts (e.g., soaps), sodium stearate, sodium lauroyl sarcosinate, perfluorononanoate, perfluorooctanoate, octenidine dihydrochloride, cetyl trimethylammonium bromide (CTAB), cetyl trimethylammonium chloride (CTAC), Cetylpyridinium chloride (CPC), polyethoxylated tallow amine (POEA), benzalkonium chloride (BAC), benzethonium chloride (BZT); 5-Bromo-5-nitro-1,3-dioxane, dimethyldioctadecylammonium chloride, dioctadecyldimethylammonium bromide, 3-[(3-Cholamidopropyl)dimethylammonio]-1-propanesulfonate (e.g., CHAPS), cocamidopropyl hydroxysultaine, amino acids, imino acids, cocamidopropyl betaine, lecithin, fatty alcohols (e.g., cetyl alcohol, stearyl alcohol, and the like), the like and combinations thereof.

A lipid molecule typically comprises at least one hydrophobic chain and at least one polar head. When exposed to an aqueous environment, lipids often will self assemble into structures that minimize the surface area exposed to a polar (e.g., aqueous) medium. Lipids sometimes assemble into structures having a single or monolayer of lipid enclosing a non-aqueous environment, and lipids sometimes assemble into structures comprising a bilayer enclosing an aqueous environment. In a monolayer structure, the polar portion of lipids (e.g., the head of the molecule in the case of phospholipids and other lipids commonly found in cell substrates) often is oriented towards the polar, aqueous environment, allowing the non-polar portion of the lipid to contact the non-polar environment.

A lipid bilayer typically comprises a sheet of lipids, generally two molecules thick, arranged so the hydrophilic phosphate heads point towards a hydrophilic aqueous environment on either side of the bilayer and the hydrophobic tails point towards the hydrophobic core of the bilayer. This arrangement results in two “leaflets” that are each a single molecular layer. Lipids self-assemble into a bilayer structure due to the hydrophobic effect and are held together entirely by non-covalent forces that do not involve formation of chemical bonds between individual molecules. Lipid bilayers generally also are impermeable to ions, which allow cells to regulate various processes that involve salt concentrations or gradients and intracellular pH by pumping ions across cell substrates using ion transport mechanisms.

In some embodiments, lipid bilayers are natural, and in certain embodiments lipid bilayers are artificially generated. Natural bilayers often are made mostly of phospholipids, which have a hydrophilic head and two hydrophobic tails (e.g., lipid tails), and form a two-layered sheet as noted above, when exposed to water or an aqueous environment. The center of this bilayer contains almost no water and also excludes molecules like sugars or salts that dissolve in water, but not in oil. Lipid tails also can affect lipid composition properties, by determining the phase of the bilayers, for example. A bilayer sometimes adopts a solid gel phase state at lower temperatures and undergoes a phase transition to a fluid state at higher temperatures. The packing of lipids within a bilayer also affects its mechanical properties, including its resistance to stretching and bending.

Artificial bilayers (e.g., sometimes also referred to as “model lipid bilayers”) are any bilayers assembled through artificial means, as opposed to bilayers that occur naturally (e.g., cell walls, lipid bilayers that cover various sub-cellular structures). An artificial bilayer can be made with synthetic and/or natural lipids, thus the process, not the material, defines an artificial or model system. Properties, such as stretching, bending or temperature induced phase transitions, have been studied with artificial model bilayers. The simplest model systems contain only a single pure synthetic lipid. The artificial bilayer also may contain a hydrophobic solvent, such as decane, hexadecane, pentane or other solvents and combinations thereof, that is used to disperse the lipid during bilayer formation and stabilize the formation of lipid bilayers across apertures in hydrophobic materials. The simplicity of a single lipid system is advantageous when determining physical or mechanical properties of bilayers. Model bilayers with greater physiological relevance can be generated utilizing mixtures of several synthetic lipids or, as mentioned, with natural lipids extracted from biological samples.

The presence of certain lipids or proteins sometimes can alter the surface chemistry of bilayers (e.g., viscosity or fluidity of lipid bilayers). Phospholipids with certain head groups can alter the surface chemistry of a bilayer. Non-limiting examples of bilayer constituents that can alter the surface chemistry of bilayers include fats, lecithin, cholesterol, proteins, phospholipids (e.g., phosphatidic acid (phosphatidate), phosphatidylethanolamine (e.g., cephalin), phosphatidylcholine (e.g., lecithin), phosphatidylserine, and phosphoinositides such as phosphatidylinositol (PI), phosphatidylinositol phosphate (PIP), phosphatidylinositol bisphosphate (PIP2) and phosphatidylinositol triphosphate (PIP3), phosphatidylglycerol, ceramide phosphorylcholine, ceramide phosphorylethanolamine, ceramide phosphorylglycerol), surfactants, the like and combinations thereof.

A device may include one or more types of molecules other than phospholipids. For example, cholesterol, which helps strengthen bilayers and decreases bilayer permeability can be included. Cholesterol also helps regulate the activity of certain integral substrate proteins. Different types or forms of lipid compositions (e.g., monolayers and/or bilayers) can be found naturally or generated artificially. Non-limiting examples of lipid compositions include monolayers (e.g., micelles) and bilayers including “black PLBs”, vesicles (e.g., sometimes referred to as “liposomes”), supported lipid bilayers, linear lipid bilayers and the like.

A nanopore membrane device often comprises a lipid composition (e.g., monolayer, bilayer, combination thereof) over, across or spanning an aperture of a substrate. A lipid composition can comprise one or more types of lipids having various chain lengths and/or various structures of polar heads. A lipid composition of a nanopore membrane device often is relatively stable to mechanical disturbances, and can have a lifetime in excess of two weeks. A lipid composition sometimes comprises a lipid monolayer, sometimes comprises a lipid bilayer, and in some embodiments comprises a lipid layer that partially is a monolayer and partially is a bilayer. A portion of a lipid composition in a device can interact with one or more exterior and/or interior surfaces of a substrate. In some devices comprising both monolayer and bilayer lipid structures, solvent may be trapped at a location (i.e., annulus) between the substrate and the lipid layer at or near the monolayer and bilayer interface, which is addressed in greater detail hereafter. In certain embodiments, a lipid composition that spans across the substrate aperture is a combination of a lipid bilayer and monolayer. In various embodiments, a lipid monolayer deposited on the exterior surface of a substrate and a lipid monolayer deposited on the interior surface of the channel or nanopore that join together at about the edge of the channel or nanopore opening can form a lipid bilayer spanning or suspended across the aperture. The bilayer formed across an aperture sometimes is referred to as a “spanning lipid bilayer” herein.

In a spanning bilayer structure, a bilayer often is present across the substrate aperture and a monolayer is present on substrate surfaces (e.g., chemically modified surfaces and/or hydrophobic). In some embodiments, a chemically modified device corrals a single protein pore in the lipid bilayer region that spans across the aperture. An inserted protein (e.g., protein pore, alpha hemolysin) often is able to diffuse in the bilayer across the aperture but often cannot leave this area to enter the lipid monolayer. Insertion of a sensing entity (e.g., protein pore) often occurs only in the bilayer region. A thin layer (e.g., about 1 to about 10 nm thick) containing solvent and ions sometimes is formed between a spanning lipid bilayer and one or more surfaces of the substrate. The thickness of this layer is defined as the distance between the exterior surface and the lipid bilayer and often plays a role in determining the resistance of the bilayer seal and the stability and fluidity of the bilayer. A spanning bilayer also sometimes includes an annulus formed between monolayers and a channel or nanopore surface, which can contain solvent (e.g., FIG. 15 of U.S. Pat. No. 7,777,505).

A protein often is inserted into a structure (e.g., monolayer and/or bilayer) formed by the lipid or amphiphilic material composition. A protein that is inserted into the structure can be water soluble, detergent-solubilized or incorporated into a lipid bilayer (e.g., vesicle, liposome) or a lipid monolayer (e.g., micelle) prior to insertion into a PLB, in some embodiments. Membrane proteins sometimes cannot be incorporated directly into the PLB during formation because immersion in an organic solvent sometimes can denature the protein. Exceptions include alpha hemolysin, MspA, and gramicidin. A membrane protein sometimes is solubilized with a detergent and added to the aqueous solution after the bilayer is formed. The dilution of the detergent stabilizing the protein forces the proteins to spontaneously insert into the bilayer over a period of minutes or hours, and often at a low frequency of success.

A vesicle is a lipid bilayer configured as a spherical shell enclosing a small amount of water or aqueous solution and separating it from the water or aqueous solution outside the vesicle. Because of the fundamental similarity to a cell wall, vesicles have been used to study the properties of lipid bilayers. Vesicles also are readily manufactured. A sample of dehydrated lipid spontaneously forms vesicles, when exposed to water. Spontaneously formed vesicles can be unilamelar (single-walled) or multilamellar (many-walled) and are of a wide range of sizes from tens of nanometers to several micrometers. A liposome is an artificially prepared vesicle, and also comprises a lipid bilayer and also can be made of naturally occurring or synthetic lipids, including phospholipids. There are four types of liposomes: MLV (multilamellar vesicles), SUV (Small Unilamellar Vesicles), LUV (Large Unilamellar Vesicles) and GUV (Giant Unilamellar Vesicles). Liposomes may be used to form PLBs on a surface or across apertures.

Unlike a vesicle or a cell substrate in which the lipid bilayer forms an enclosed shell, a supported bilayer (e.g., SLB) is a planar structure in contact with a substrate. One advantage of the supported bilayer is its stability. SLBs often remain largely intact even when subject to high flow rates or vibration, and the presence of holes will not destroy the entire bilayer. Due to the stability of SLB's, experiments lasting weeks and even months can be conducted with supported bilayers, while BLM experiments sometimes are limited to hours. Another advantage of the supported bilayer is the greater number of methods and tools useable for characterization. In certain embodiments, a substrate may comprise a hydrophilic material, such as untreated glass, or it may be modified in a manner that renders one or more surfaces of the substrate (e.g., pore interior, pore exterior) hydrophilic (e.g. mildly hydrophilic, substantially hydrophilic). In certain embodiments, the bilayer is then formed over the hydrophilic surface and covers across the substrate aperture.

In certain embodiments, a substrate may include a hydrophobic material, such as Teflon, or it may be modified in a manner that renders one or more surfaces of the substrate (e.g., substrate channel interior, substrate channel exterior) hydrophobic (e.g. mildly hydrophobic, substantially hydrophobic). In some embodiments one or more surfaces of a substrate are coated with a hydrophobic substance, including without limitation an alkyl silane substance (e.g., 3-cyano-propyldimethylchlorosilane). Any suitable silane substance can be selected to render a substrate surface more hydrophobic and support interaction with lipids for formation of a lipid structure that spans the substrate aperture. In some embodiments, a spanning lipid structure contains a monolayer that interacts with an exterior surface of a substrate and a monolayer that interacts with an interior surface of the substrate, where the monolayers join together at about the edge of the opening of the aperture and form a lipid bilayer spanning the substrate aperture (e.g., U.S. Pat. No. 7,777,505, entitled “Nanopore platforms for ion channel recordings and single molecule detection and analysis,” naming White et al. as inventors).

In certain embodiments, a nanopore apparatus comprises a Nanopore Membrane System as described in U.S. patent application Ser. No. 13/414,636 filed on Mar. 7, 2012, entitled “METHODS FOR VOLTAGE-INDUCED PROTEIN INCORPORATION INTO PLANAR LIPID BILAYERS,” naming Ryan Dunnam, Geoffrey Barran and Melissa Poquette as inventors, and designated by attorney docket no. EBS-1002-UT, the entirety of which herein is incorporated by reference, including all text, tables and drawings.

The following Examples illustrate but do not limit the technology described herein.

### EXAMPLES

### Example 1

### Characterization of Alpha Hemolysin Nanopores

The following example shows the characterization of numerous alpha hemolysin nanopores, the majority with at least one mutagenesis modification, using a computed CCNR value. The CCNR was measured using immobilized single stranded DNA, polyA40 (SEQ ID NO: 7) and polyC40 (SEQ ID NO: 3). In this example, the first level of the first composition section of the first polymer was I_A40 (“A40” disclosed as SEQ ID NO: 7) of the polyA40 (SEQ ID NO: 7) polymer and the second level of the second composition section of the second polymer was I_C40 (“C40” disclosed as SEQ ID NO: 3) of the polyC40 (SEQ ID NO: 3) polymer. In this example, the levels were averaged over many measurements. The noise was computed as the square root of the sum of squares of the standard deviation of the residual currents I_A40 (“A40” disclosed as SEQ ID NO: 7) and I_C40 (“C40” disclosed as SEQ ID NO: 3) at a set filter frequency of 300 Hz. Nanopores were characterized by screening the nanopores and nanopores that met a set threshold of the CCNR of 15 at 300 Hz were put through additional testing.

### Apparatus

Glass nanopore membranes (GNMs) (as described in U.S. Pat. No. 7,777,505) were fabricated from soda lime glass or quartz as described by Zhang (B. Zhang, J. Galusha, P. G. Shiozawa et al., “Bench-Top Method for Fabricating Glass-Sealed Nanodisk Electrodes, Glass Nanopore Electrodes, and Glass Nanopore Membranes of Controlled Size,” Anal. Chem., vol. 79, no. 13, pp. 4778-4787, 2007) with radii between 500 nanometers (nm) and 1000 nm. The interior of the GNM was filled with an electrolyte solution of 3 Molar (M) NaCl, 10 milliMolar (mM) Tris, 1 mM EDTA and pH 7.1 and was inserted horizontally through the wall of a polycarbonate cell into a fluid reservoir. A Ag/AgCl electrode was produced by treating a 0.25 millimeter (mm) diameter silver wire with household bleach and was placed interior to the GNM. A pipette holder provided a secure mounting for the GNM and Ag/AgCl electrode interior to the GNM, and a means of maintaining a constant back pressure from 0 to 200 mmHg on the GNM. The test cell had a reservoir of 250 microliters and inlet/outlet ports connected to syringes to allow for raising and lowering the fluid level in the reservoir. A second Ag/AgCl sintered disk electrode served as the reference electrode and was located in the test cell reservoir. The test cell reservoir was defined as the cis side and the interior of the GNM was defined as the trans side.

The data was collected using a DC measurement system. The GNM electrode and reference electrode were connected to a custom resistive feedback headstage (F. J. Sigworth, “Design of the Patch Clamp,” Single-channel recording, B. Sakmann and E. Neher, eds., pp. 3-35, New York: Plenum Press, 1995) that allows for applying a voltage bias between the electrodes and provides a low noise readout of the current between the two electrodes. The readout amplifier employed a feedback composed of a 10 gigaohms resistor in parallel with a capacitance of approximately 1 picoFarad (pF). All voltages were referenced with respect to the electrode in the GNM. For example, a negative bias indicated that the test cell reservoir electrode was at a negative potential with respect to the electrode interior to the GNM. The output voltage of the amplifier was digitized at a rate of 1.25 MHz using a PCI-6251 DAQ card (National Instruments) in a personal computer (Dell). The resulting data was filtered using an 8-pole Bessel filter at 50 kHz, the effective bandwidth, and down sampled to 250 kHz, the sampling frequency. A final digital differentiation step then converted the filtered signal to the current between the electrodes. The same PCI card was used to provide control of the voltage bias across the two electrodes. A custom LabView application handled voltage control, data acquisition, and simple signal processing such as filtering and conversion to current.

### Bilayer Formation

1,2-diphytanoyl-sn-glycero-3-phosphocholine (DPhPC) was diluted in decane to a concentration of 5 milligrams/milliliter (mg/mL). A small (<0.5 microliters) drop of the lipid/decane mixture was added to the surface of electrolyte. The fluid level in the test cell reservoir was lowered below the face of the GNM and then raised above the face of the GNM. This action typically resulted in a bilayer, although in some cases additional lipid was added and the raising and lowering repeated. The bilayer formation method is detailed in U.S. patent application Ser. No. 12/325,792 and is herein incorporated by reference.

### Alpha Hemolysin Pore Preparation and Incorporation

Monomeric wild type alpha hemolysin (List Laboratories) was hydrated in 18.2 megaohm-cm water (Thermo-Scientific) to a produce a stock solution of 1 mg/mL. Aliquots were then diluted to 0.1 mg/mL from which about 0.1 microliter was added to the test cell for each experiment utilizing the wild type pore.

Alpha hemolysin protein monomers were generated through coupled in vitro transcription and translation (IVTT) using a bacterial extract kit (Promega) and then assembled into homo-heptamers on rabbit red blood cell membranes (rRBCM) based on established protocols (B. Walker, and H. Bayley, “Key Residues for Membrane Binding, Oligomerization, and Pore Forming Activity of Staphylococcal alpha-Hemolysin Identified by Cysteine Scanning Mutagenesis and Targeted Chemical Modification,” J. Biol. Chem., vol. 270, no. 39, pp. 23065-23071, Sep. 29, 1995, 1995). Plasmid DNA (>95% supercoiled) of wild type and mutant alpha hemolysin were made by GenScript. For most IVTT reactions, 4 micrograms of the DNA (Genscript) were mixed with contents of the kit according to the manufacturer's recommendation and supplemented with a mixture of a complete set of amino acids and 4 microCi of S³⁵-Methionine (American Radiolabeled Chemicals). The mixture was incubated at 37° C. for one hour, then mixed with rRBCM and further incubated for three hours. At the end of the incubation period, membranes were washed twice with MOPS buffer followed by solubilization with SDS loading buffer. The latter was loaded onto a 5% polyacrylamide gel and proteins separated by applying a 60 V voltage overnight at room temperature. Gels were dried under vacuum at 60° C. for 3-4 hours and exposed to X-ray film (Kodak) overnight at −80° C. Gels were developed manually using Kodak Development and Wash solutions. Bands corresponding to alpha hemolysin were observed on the developed film due to the incorporation of the radioactive methioine. The film was used as a template to cut out portion of the dried gel containing the αHL protein. Proteins were recovered from this portion by overnight electro-elution using an Elutrap Electroelution system (GE Healthcare) and concentrated down to a volume of 10-20 microL using microfuge concentrators (Millipore). Proteins were stored at −80° C. until use. Numerous mutated nanopores (>100) were made according to this protocol and their CCNR subsequently computed.

Alpha hemolysin incorporation in the bilayer was achieved by applying a back pressure (10-200 mmHg) to the interior of the GNM relative to the test cell reservoir. The precise pressure applied was determined by measuring the pressure at which the bilayer fails and using a pressure about 10 mmHg lower. After a single alpha hemolysin protein pore was incorporated as determined by a large jump in conducted current, the pressure was reduced to maintain a single protein insertion. This holding pressure was determined by measuring the pressure at which alpha hemolysin was forced out of the bilayer. In some cases, the protein concentration was too low to allow for incorporation by applying a back pressure alone. In this case a high bias (>200 mV) was applied across the bilayer to promote protein insertion, as described in a recently filed U.S. provisional application by EBS, U.S. No. 61/450,475.

### Polymers—Immobilized Single Stranded DNA

In this example, the polymers were biotinylated, single stranded homo-oligomers, Btn-5′-polyA40-3′ (SEQ ID NO: 7) and Btn-5′-polyC40-3′ (SEQ ID NO: 3), (Sigma) and they were PAGE purified and delivered in 10 milliMolar Tris, 1 milliMolar EDTA at a concentration of 20 microMolar. These solutions were stored at −80° C. for future use. Prior to immobilizing the single stranded DNA in the nanopore, the biotin (BTN) capped ssDNA sample was mixed with streptavadin (100 microM in 18.2 megaohm-cm water) at a molar ratio of 1:2.5. Following a waiting period of at least 15 minutes, about 4.0 microL of the ssDNA-streptavidin solution was added to the test cell to achieve a final DNA concentration of about 0.25 microM. A negative bias of −120 mV was applied until a single streptavidin bound DNA was captured in the alpha hemolysin pore. Once captured, the DNA was electrophoretically trapped within the alpha hemolysin pore for a period of 0.5 to 2 seconds and residual current measurements were recorded. After which, the applied negative bias was inverted for a period of 0.02 to 1 s, in order to drive the DNA out of the pore. The applied bias was then switched back to −120 mV in order to reset the capture experiment. This capture and release routine was carried out hundreds of times in order to acquire adequate statistics of the captured DNA residual current and associated noise level. After acquiring data with a single ssDNA sample (e.g. polyA40 (SEQ ID NO: 7)), a second sample was added to the solution (e.g. polyC40 (SEQ ID NO: 3)) and the experiment was carried out a second time in order to acquire captured DNA residual current statistics and associated noise levels for the second DNA sample. This also allowed for direct comparison between the two DNA samples residual currents and noise levels and a direct means for determining the CCNR for various nucleotides in a given mutant HL pore.

### Data Analysis

Immobilized events were idealized using the segmental k-means algorithm included in the QuB software package (University of Buffalo). Idealized event data including the event duration, amplitude and noise level were exported for further analysis by custom Python software. Computation of noise power spectral density and event statistics was performed using the Scientific and Numerical Python packages, SciPy and Numpy.

### Results and Discussion

### DNA Immobilization

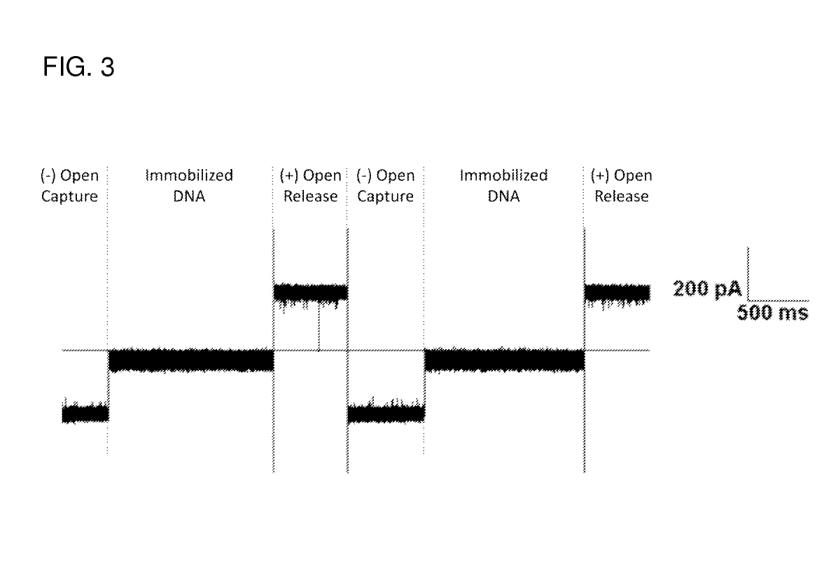

An example data set from the ssDNA immobilization or “Capture and Release” experiment was shown in FIG. 3. The bandwidth equals 100 kHz. The ssDNA was biotinylated on the 5′ end and attached to streptavidin to prevent translocation. The DNA was driven into the cis side of the pore under a negative bias. Under an applied bias of −120 mV, the ssDNA was captured and held immobilized for 0.5 to 2 seconds. The voltage was then reversed to +120 mV to release the ssDNA from the pore. The process was then repeated depending upon the needs of the individual experiment.

A relatively rapid approach to screening pores for improved CCNR has been used to characterize nanopores to determine promising pores for nanopore sequencing. A large number (10's to 100's) of capture and release measurements were made for one type of ssDNA (e.g. Btn-5′-polyA40-3′ (SEQ ID NO: 7)). A second type of ssDNA (e.g. Btn-5′-polyC40-3′ (SEQ ID NO: 3)) was then added and a large number of captures were performed with both ssDNA types present. From each of the immobilization events, the residual current and noise level was measured. By measuring the blockades of one DNA type initially and then the mixture, it was possible to eliminate other factors that might produce an apparent contrast between the nucleotides. In particular, changes in temperature, the electrolyte concentration and even the specific protein inserted can produce residual current differences as large as the contrast to be measured.

### Characterization

A large number of pores have been evaluated with modifications concentrated on the primary and secondary constrictions within the alpha hemolysin pore. The majority of the modifications yielded proteins that could be successfully inserted into planar lipid bilayers. In this example, those that were found to be viable, the contrast between Btn-5′-polyA40-3′ (SEQ ID NO: 7) and Btn-5′-polyC40-3′ (SEQ ID NO: 3) was measured as well as the root mean square (RMS) noise level for each polymer. The noise level was measured following the application of a 300 Hz low pass filter in order to eliminate any potential contribution from changes in capacitance and to reduce the effect of low frequency electrical and vibration interference near 60 Hz. Assuming noise power was relatively flat with frequency between 100 Hz and 10 kHz, the 300 Hz filtered noise can be scaled by the square root of (10,000/300) to estimate the noise at 10 kHz, which is a bandwidth value that is more likely to be used in a polymer sequencing system.

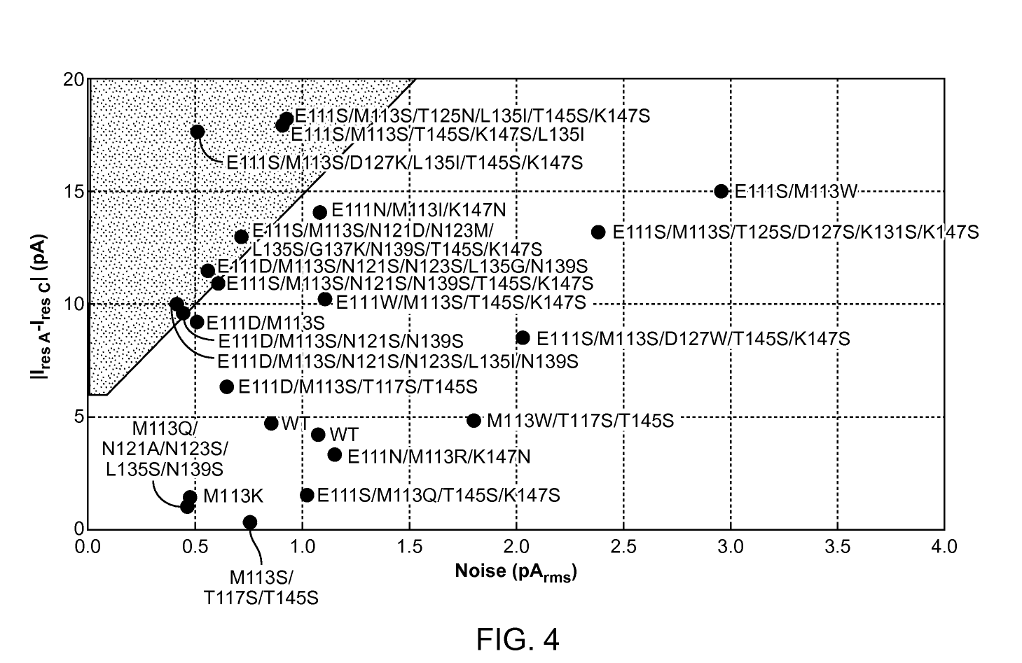

The resulting contrast and noise data are shown in FIG. 4 and are summarized in Table 3. In this example, the contrast signal was the magnitude of the difference between the residual current with polyA40 (SEQ ID NO: 7) and polyC40 (SEQ ID NO: 3) in the pore. By taking the magnitude of the contrast, the distinction between greater blocking by A or C was removed and both cases were more directly compared. In this example, the noise level was computed from the square root of the sum of the squares of the standard deviations of the residual current levels at the 300 Hz set filter frequency (e.g. a 300 Hz low pass, 3-pole Bessel filter) for the polyA40 (SEQ ID NO: 7) and polyC40 (SEQ ID NO: 3). Each point was labeled with the specific alpha hemolysin mutation. For example, T145S was the mutation wherein the threonine at residue 145 has been changed to serine for all of the 7 chains in the pore. A double mutation was represented by each individual mutation separated by a forward slash (e.g. E111S/M113S). The noise level was computed after filtering the data. The shaded region in FIG. 4 designates a CCNR value of 15 at a set filter frequency of 300 Hz. In TABLE 3, data in the shaded boxes indicates the nanopore met the CCNR threshold of 15 at a set filter frequency of 300 Hz.

TABLE 3

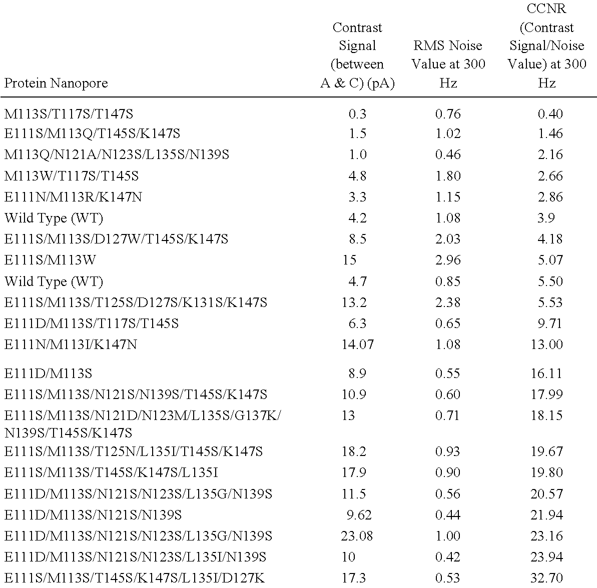

The amino acid sequence for wild type alpha hemolysin is provided in SEQ ID NO. 1 for reference:

SEQ ID NO: 1
MADSDINIKTGTTDIGSNTTVKTGDLVTYDKENGMHKKVFYSFIDDKNHNKKLLVIRTKGTIAGQYRVYSEEGA
NKSGLAWPSAFKVQLQLPDNEVAQISDYYPRNSIDTKEYMSTLTYGFNGNVTGDDTGKIGGLIGANVSIGHTLK
YVQPDFKTILESPTDKKVGWKVIFNNMVNQNWGPYDRDSWNPVYGNQLFMKTRNGSMKAADNFLDPNKASSLLS
SGFSPDFATVITMDRKASKQQTNIDVIYERVRDDYQLHWTSTNWKGTNTKDKWTDRSSERYKIDWEKEEMTN

The contrast to noise chart provides a variety of information. The CCNR was computed by dividing the contrast signal by the RMS noise value. CCNR for the contrast measurement was effectively determined by the slope of the line from any given data point through the origin. The line in the plot has a slope equal to the average CCNR measured for wild type αHL (WT). Points that lie well above the line, such as that for E111N/M113I/K147N, have a relatively high CCNR whereas those below have a relatively low CCNR. The contrast versus noise chart also makes it clear that a high contrast alone does not guarantee a high CCNR. The mutation E111S/M113W is a particularly good example. This mutation has a high contrast, but the noise level was also very high. As a result, this mutant has an SNR of 4.9, which is only marginally better than WT. E111D/M113S has a contrast of only 8.9 pA, significantly less than the contrast of E111S/M113W, but the CNR for E111D/M113S was relatively high at 16.11. This data reinforces the conclusion that both the contrast and the noise must be considered in order to identify mutations that are suitable for nanopore sequencing.

This plot shows that the targeted mutations involving the primary and secondary constrictions as well as the salt bridges can have a significant effect on the CCNR.

In this example, the nanopores were characterized by screening and selecting nanopores based on the computed CCNR being above a set threshold. The set threshold was 15 at 300 Hz. Nanopores that met or exceeded this threshold underwent additional testing such as mapping.

### Example 2

### Examples of Embodiments

Provided hereafter are non-limiting examples of certain embodiments of the technology.

A1. A method for characterizing a nanopore based on a characterization contrast signal to noise ratio (CCNR) comprising:

measuring a first level within a residual current for a first composition section of a polymer;

measuring a second level within a residual current for a second composition section of a polymer;

calculating a contrast signal as a function of the first level and the second level; computing a noise value;

calculating a characterization contrast signal to noise ratio (CCNR) as a function of the

contrast signal and the noise value; and

characterizing the nanopore based on the CCNR.

A2. The method of embodiment A1, wherein a first polymer comprises the first composition section and the second composition section.
A3. The method of embodiment A1, wherein a first polymer comprises the first composition section and a second polymer comprises the second composition section.
A4. The method of any one of embodiments A1 to A3, wherein the pore of the nanopore comprises a beta barrel.
A5. The method of embodiment A4, wherein the nanopore is chosen from alpha hemolysin, MspA, OmpF, PA63 and gramicidin A.
A6. The method of embodiment A5, wherein the nanopore is an alpha hemolysin.
A7. The method of any one of embodiments A1 to A6, wherein each polymer is a protein or peptide.
A7.1. The method of any one of embodiments A1 to A6, wherein each polymer is a nucleic acid.
A7.2. The method of embodiment A7.1, wherein the nucleic acid is chosen from DNA and RNA.
A7.3. The method of embodiment A7.1 or A7.2, wherein the nucleic acid is single stranded or double stranded.
A8. The method of embodiment A7.1, wherein the polymer is single stranded DNA.
A9. The method of any one of embodiments A1 to A8, wherein the composition section comprises at least a part of a monomer.
A10. The method of embodiment A9, wherein the monomer independently is chosen from a nucleotide, monophosphate nucleotide, oxidized nucleotide, methylated nucleotide and modified nucleotide.
A11. The method of embodiment A10, wherein the nucleotide comprises a base chosen from adenine, cytosine, thymine, guanine and uracil.
A12. The method of any one of embodiments A1 to A11, wherein each polymer is immobilized within the nanopore while the residual current is measured.
A13. The method of embodiment A12, wherein each polymer is immobilized within the nanopore by attaching at least one molecule to the polymer.
A14. The method of embodiment A13, wherein the at least one molecule attached to each polymer is chosen from biotin, tetraethylene glycol, avidin, streptavidin, Neutravidin and combinations thereof.
A15. The method of embodiment A12, wherein each polymer is immobilized within the nanopore by a double stranded nucleic acid.
A16. The method of embodiment A15, wherein a portion of or all of each polymer is double stranded.
A17. The method of embodiment A15, wherein the double stranded nucleic acid comprises a hairpin.
A18. The method of embodiment A15, wherein the double stranded nucleic acid comprises a complementary strand of single stranded nucleic acid hybridized to a portion of each polymer.
A19. The method of any one of embodiments A1 to A18, wherein each polymer is within the nanopore and translocates the nanopore while the residual current is measured.
A20. The method of embodiment A19, wherein the first level is correlated to the composition of the first composition section of the polymer.
A21. The method of embodiment A19 or A20, wherein the second level is correlated to the composition of the second composition section of the polymer.
A22. The method of any one of embodiments A19 to A21, wherein the level is correlated to the composition of the composition section of the polymer using homopolymers that are translocating the nanopore.
A23. The method of embodiments A19 to A21, wherein the level is correlated to the composition of the composition section of the polymer by mapping the nanopore with a heteropolymer.
A24. The method of any one of embodiments A1 to A23, wherein calculating the contrast signal comprises determining the difference between a single measurement of the first level and a single measurement of the second level.
A25. The method of any one of embodiments A1 to A23, wherein calculating the contrast signal comprises determining the difference between an average of the measurements of the first level and an average of the measurements of the second level.
A26. The method of embodiment A25, wherein the average of the measurements of the first level is the average of at least 20 measurements of the first level.
A27. The method of embodiment A25 or A26, wherein the average of the measurements of the second level is the average of at least 20 measurements of the second level.
A28. The method of any one of embodiments A1 to A27, wherein the noise value is the larger value of the noise computed for the first level and the noise computed for the second level.
A29. The method of any one of embodiments A1 to A27, wherein the noise value is the smaller value of the noise computed for the first level and the noise computed for the second level.
A30. The method of any one of embodiments A1 to A29, wherein the noise level is computed for the first level and the second level.
A31. The method of any one of embodiments A1 to A30, wherein computing the noise value comprises using the noise amplitude at a given frequency.
A32. The method of any one of embodiments A1 to A31, wherein computing the noise value comprises using the root mean square noise value of the first level and the second level at a set filter frequency.
A33. The method of any one of embodiments A1 to A32, wherein the noise value is the square root of the sum of the squares of the standard deviation value for the first level and the standard deviation value for the second level at a set filter frequency.
A34. The method of any one of embodiments A1 to A33, wherein the noise value is extracted from the noise power spectral density.
A35. The method of any one of embodiments A1 to A34, wherein the CCNR is calculated by dividing the contrast signal by the noise value.
A36. The method of any one of embodiments A1 to A35, wherein the residual current measurements are acquired using a Direct Current (DC) measurement system.
A37. The method of any one of embodiments A1 to A36, wherein the residual current measurements are acquired using an Alternating Current (AC) or an AC/DC (Direct Current) measurement system.
A38. The method of any one of embodiments A1 to A37, wherein data is filtered to a set filter frequency using a low pass filter.
A39. The method of embodiment A38, wherein the data is filtered to a set filter frequency using a 3-pole Bessel low pass filter.
A40. The method of any one of embodiments A1 to A39, wherein the set filter frequency is chosen from about 1 Hz to about 500 kHz.
A41. The method of embodiment A40, wherein the set filter frequency is about 300 Hz.
A42. The method of embodiment A40, wherein the set filter frequency is about 10 kHz.
A43. The method of any one of embodiments A1 to A42, wherein characterizing the nanopore comprises comparing the CCNR calculated to a threshold CCNR.
A44. The method of embodiment A43, wherein the set threshold CCNR is 2.
A45. The method of embodiment A44, wherein the threshold CCNR is 5.
A46. The method of any one of embodiments A43 to A45, wherein a nanopore for which a calculated CCNR is equal to or greater than the threshold CCNR is subjected to further testing.
A47. The method of embodiment A46, wherein the further testing comprises mapping the nanopore.
A48. The method of embodiment A47, wherein the mapping comprises contacting the nanopore with heteropolymers.
A49. The method of embodiment A48, wherein the heteropolymers comprise a particular nucleotide or nucleotide sequence at a different position in each of the heteropolymers.
A50. The method of embodiment A48 or A49, wherein the mapping comprises computing the CCNR for the heteropolymers.
A51. The method of any one of embodiments A1 to A50, wherein characterizing the nanopore comprises determining the effect of a mutagenesis modification on the CCNR computed for the nanopore.
A52. The method of any one of embodiments A1 to A51, wherein characterizing the nanopore comprises identifying one or more further mutagenesis modifications that can be made to the nanopore.
A53. The method of any one of embodiments A1 to A52, wherein characterizing the nanopore comprises selecting a nanopore that can determine the sequence of a polymer.
A54. The method of any one of embodiments A1 to A51, wherein the polymer is a nucleic acid.
B1. A device comprising a nanopore characterized by any one of the methods of embodiments A1 to A54.

The entirety of each patent, patent application, publication and document referenced herein hereby is incorporated by reference. Citation of the above patents, patent applications, publications and documents is not an admission that any of the foregoing is pertinent prior art, nor does it constitute any admission as to the contents or date of these publications or documents. Modifications may be made to the foregoing without departing from the basic aspects of the technology. Although the technology has been described in substantial detail with reference to one or more specific embodiments, those of ordinary skill in the art will recognize that changes may be made to the embodiments specifically disclosed in this application, yet these modifications and improvements are within the scope and spirit of the technology.

The technology illustratively described herein suitably may be practiced in the absence of any element(s) not specifically disclosed herein. Thus, for example, in each instance herein any of the terms “comprising,” “consisting essentially of,” and “consisting of” may be replaced with either of the other two terms. The terms and expressions which have been employed are used as terms of description and not of limitation, and use of such terms and expressions do not exclude any equivalents of the features shown and described or portions thereof, and various modifications are possible within the scope of the technology claimed. The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 10%), and use of the term “about” at the beginning of a string of values modifies each of the values (i.e., “about 1, 2 and 3” refers to about 1, about 2 and about 3). For example, a weight of “about 100 grams” can include weights between 90 grams and 110 grams. Further, when a listing of values is described herein (e.g., about 50%, 60%, 70%, 80%, 85% or 86%) the listing includes all intermediate and fractional values thereof (e.g., 54%, 85.4%). Thus, it should be understood that although the present technology has been specifically disclosed by representative embodiments and optional features, modification and variation of the concepts herein disclosed may be resorted to by those skilled in the art, and such modifications and variations are considered within the scope of this technology.

Certain embodiments of the technology are set forth in the claim(s) that follow(s).

1. A method for characterizing a nanopore based on a characterization contrast signal to noise ratio (CCNR) comprising:
measuring a first level within a residual current for a first composition section of a polymer;
measuring a second level within a residual current for a second composition section of a polymer;
calculating a contrast signal as a function of the first level and the second level;
computing a noise value;
calculating a characterization contrast signal to noise ratio (CCNR) as a function of the contrast signal and the noise value; and
characterizing the nanopore based on the CCNR.

2. The method of claim 1, wherein a first polymer comprises the first composition section and the second composition section.

3. The method of claim 1, wherein a first polymer comprises the first composition section and a second polymer comprises the second composition section.

4-5. (canceled)

6. The method of claim 1, wherein the nanopore is an alpha hemolysin.

7. (canceled)

8. The method of claim 1, wherein the polymer is a nucleic acid.

9. The method of claim 8, wherein the nucleic acid is DNA or RNA.

10. (canceled)

11. The method of claim 8, wherein the polymer is single stranded DNA.

12-14. (canceled)

15. The method of claim 1, wherein each polymer is immobilized within the nanopore while the residual current is measured.

16-21. (canceled)

22. The method of claim 1, wherein each polymer is within the nanopore and translocates the nanopore while the residual current is measured.

23-45. (canceled)

46. The method of claim 1, wherein characterizing the nanopore comprises comparing the CCNR calculated to a threshold CCNR.

47. The method of claim 46, wherein the set threshold CCNR is 2.

48. The method of claim 46, wherein the threshold CCNR is 5.

49. The method of claim 46, wherein a nanopore for which a calculated CCNR is equal to or greater than the threshold CCNR is subjected to further testing.

50. The method of claim 49, wherein the further testing comprises mapping the nanopore.

51. The method of claim 50, wherein the mapping comprises contacting the nanopore with heteropolymers.

52. The method of claim 51, wherein the heteropolymers comprise a particular nucleotide or nucleotide sequence at a different position in each of the heteropolymers.

53. The method of claim 51 or 52, wherein the mapping comprises computing the CCNR for the heteropolymers.

54. The method of claim 1, wherein characterizing the nanopore comprises determining the effect of a mutagenesis modification on the CCNR computed for the nanopore.

55. The method of claim 1, wherein characterizing the nanopore comprises identifying one or more further mutagenesis modifications that can be made to the nanopore.

56. The method of claim 1, wherein characterizing the nanopore comprises selecting a nanopore that can determine the sequence of a polymer.

57. (canceled)

58. A device comprising a nanopore characterized by the method of claim 1.
